SEQUENCE LISTING (7 sequences)

SEQ ID NO: 1 - DNA, 4113 bp, Homo sapiens
  CDS (22)...(4110)
acccacgcgcagcggccggagatgcagcggggcgccgcgctgtgcctgcgactgtggctctgcctgggactcctggacggcctggtgagtgactactccatgacccccccgaccttgaacatcacggaggagtcacacgtcatcgacaccggtgacagcctgtccatctcctgcaggggacagcaccccctcgagtgggcttggccaggagctcaggaggcgccagccaccggagacaaggacagcgaggacacgggggtggtgcgagactgcgagggcacagacgccaggccctactgcaaggtgttgctgctgcacgaggtacatgccaacgacacaggcagctacgtctgctactacaagtacatcaaggcacgcatcgagggcaccacggccgccagctcctacgtgttcgtgagagactttgagcagccattcatcaacaagcctgacacgctcttggtcaacaggaaggacgccatgtgggtgccctgtctggtgtccatccccggcctcaatgtcacgctgcgctcgcaaagctcggtgctgtggccagacgggcaggaggtggtgtgggatgaccggcggggcatgctcgtgtccacgccactgctgcacgatgccctgtacctgcagtgcgagaccacctggggagaccaggacttcctttccaaccccttcctggtgcacatcacaggcaacgagctctatgacatccagctgttgcccaggaagtcgctggagctgctggtaggggagaagctggtcctcaactgcaccgtgtgggctgagtttaactcaggtgtcacctttgactgggactacccagggaagcaggcagagcggggtaagtgggtgcccgagcgacgctcccaacagacccacacagaactctccagcatcctgaccatccacaacgtcagccagcacgacctgggctcgtatgtgtgcaaggccaacaacggcatccagcgatttcgggagagcaccgaggtcattgtgcatgaaaatcccttcatcagcgtcgagtggctcaaaggacccatcctggaggccacggcaggagacgagctggtgaagctgcccgtgaagctggcagcgtaccccccgcccgagttccagtggtacaaggatggaaaggcactgtccgggcgccacagtccacatgccctggtgctcaaggaggtgacagaggccagcacaggcacctacaccctcgccctgtggaactccgctgctggcctgaggcgcaacatcagcctggagctggtggtgaatgtgcccccccagatacatgagaaggaggcctcctcccccagcatctactcgcgtcacagccgccaggccctcacctgcacggcctacggggtgcccctgcctctcagcatccagtggcactggcggccctggacaccctgcaagatgtttgcccagcgtagtctccggcggcggcagcagcaagacctcatgccacagtgccgtgactggagggcggtgaccacgcaggatgccgtgaaccccatcgagagcctggacacctggaccgagtttgtggagggaaagaataagactgtgagcaagctggtgatccagaatgccaacgtgtctgccatgtacaagtgtgtggtctccaacaaggtgggccaggatgagcggctcatctacttctatgtgaccaccatccccgacggcttcaccatcgaatccaagccatccgaggagctactagagggccagccggtgctcctgagctgccaagccgacagctacaagtacgagcatctgcgctggtaccgcctcaacctgtccacgctgcacgatgcgcacgggaacccgcttctgctcgactgcaagaacgtgcatctgttcgccacccctctggccgccagcctggaggaggtggcacctggggcgcgccacgccacgctcagcctgagtatcccccgcgtcgcgcccgagcacgagggccactatgtgtgcgaagtgcaagaccggcgcagccatgacaagcactgccacaagaagtacctgtcggtgcaggccctggaagcccctcggctcacgcagaacttgaccgacctcctggtgaacgtgagcgactcgctggagatgcagtgcttggtggccggagcgcacgcgcccagcatcgtgtggtacaaagacgagaggctgctggaggaaaagtctggagtcgacttggcggactccaaccagaagctgagcatccagcgcgtgcgcgaggaggatgcgggaccgtatctgtgcagcgtgtgcagacccaagggctgcgtcaactcctccgccagcgtggccgtggaaggctccgaggataagggcagcatggagatcgtgatccttgtcggtaccggcgtcatcgctgtcttcttctgggtcctcctcctcctcatcttctgtaacatgaggaggccggcccacgcagacatcaagacgggctacctgtccatcatcatggaccccggggaggtgcctctggaggagcaatgcgaatacctgtcctacgatgccagccagtgggaattcccccgagagcggctgcacctggggagagtgctcggctacggcgccttcgggaaggtggtggaagcctccgctttcggcatccacaagggcagcagctgtgacaccgtggccgtgaaaatgctgaaagagggcgccacggccagcgagcagcgcgcgctgatgtcggagctcaagatcctcattcacatcggcaaccacctcaacgtggtcaacctcctcggggcgtgcaccaagccgcagggccccctcatggtgatcgtggagttctgcaagtacggcaacctctccaacttcctgcgcgccaagcgggacgccttcagcccctgcgcggagaagtctcccgagcagcgcggacgcttccgcgccatggtggagctcgccaggctggatcggaggcggccggggagcagcgacagggtcctcttcgcgcggttctcgaagaccgagggcggagcgaggcgggcttctccagaccaagaagctgaggacctgtggctgagcccgctgaccatggaagatcttgtctgctacagcttccaggtggccagagggatggagttcctggcttcccgaaagtgcatccacagagacctggctgctcggaacattctgctgtcggaaagcgacgtggtgaagatctgtgactttggccttgcccgggacatctacaaagaccccgactacgtccgcaagggcagtgcccggctgcccctgaagtggatggcccctgaaagcatcttcgacaaggtgtacaccacgcagagtgacgtgtggtcctttggggtgcttctctgggagatcttctctctgggggcctccccgtaccctggggtgcagatcaatgaggagttctgccagcgcgtgagagacggcacaaggatgagggccccggagctggccactcccgccatacgccacatcatgctgaactgctggtccggagaccccaaggcgagacctgcattctcggacctggtggagatcctgggggacctgctccagggcaggggcctgcaagaggaagaggaggtctgcatggccccgcgcagctctcagagctcagaagagggcagcttctcgcaggtgtccaccatggccctacacatcgcccaggctgacgctgaggacagcccgccaagcctgcagcgccacagcctggccgccaggtattacaactgggtgtcctttcccgggtgcctggccagaggggctgagacccgtggttcctccaggatgaagacatttgaggaattccccatgaccccaacgacctacaaaggctctgtggacaaccagacagacagtgggatggtgctggcctcggaggagtttgagcagatagagagcaggcatagacaagaaagcggcttcagctgtaaaggacctggccagaatgtggctgtgaccagggcacaccctgactcccaagggaggcggcggcggcctgagcggggggcccgaggaggccaggtgttttacaacagcgagtatggggagctgtcggagccaagcgaggaggaccactgctccccgtctgcccgcgtgactttcttcacagacaacagctactaa

SEQ ID NO: 2 - protein, 1363 aa, Homo sapiens
MQRGAALCLRLWLCLGLLDGLVSDYSMTPPTLNITEESHVIDTGDSLSISCRGQHPLEWAWPGAQEAPATGDKDSEDTGVVRDCEGTDARPYCKVLLLHEVHANDTGSYVCYYKYIKARIEGTTAASSYVFVRDFEQPFINKPDTLLVNRKDAMWVPCLVSIPGLNVTLRSQSSVLWPDGQEVVWDDRRGMLVSTPLLHDALYLQCETTWGDQDFLSNPFLVHITGNELYDIQLLPRKSLELLVGEKLVLNCTVWAEFNSGVTFDWDYPGKQAERGKWVPERRSQQTHTELSSILTIHNVSQHDLGSYVCKANNGIQRFRESTEVIVHENPFISVEWLKGPILEATAGDELVKLPVKLAAYPPPEFQWYKDGKALSGRHSPHALVLKEVTEASTGTYTLALWNSAAGLRRNISLELVVNVPPQIHEKEASSPSIYSRHSRQALTCTAYGVPLPLSIQWHWRPWTPCKMFAQRSLRRRQQQDLMPQCRDWRAVTTQDAVNPIESLDTWTEFVEGKNKTVSKLVIQNANVSAMYKCVVSNKVGQDERLIYFYVTTIPDGFTIESKPSEELLEGQPVLLSCQADSYKYEHLRWYRLNLSTLHDAHGNPLLLDCKNVHLFATPLAASLEEVAPGARHATLSLSIPRVAPEHEGHYVCEVQDRRSHDKHCHKKYLSVQALEAPRLTQNLTDLLVNVSDSLEMQCLVAGAHAPSIVWYKDERLLEEKSGVDLADSNQKLSIQRVREEDAGPYLCSVCRPKGCVNSSASVAVEGSEDKGSMEIVILVGTGVIAVFFWVLLLLIFCNMRRPAHADIKTGYLSIIMDPGEVPLEEQCEYLSYDASQWEFPRERLHLGRVLGYGAFGKVVEASAFGIHKGSSCDTVAVKMLKEGATASEQRALMSELKILIHIGNHLNVVNLLGACTKPQGPLMVIVEFCKYGNLSNFLRAKRDAFSPCAEKSPEQRGRFRAMVELARLDRRRPGSSDRVLFARFSKTEGGARRASPDQEAEDLWLSPLTMEDLVCYSFQVARGMEFLASRKCIHRDLAARNILLSESDVVKICDFGLARDIYKDPDYVRKGSARLPLKWMAPESIFDKVYTTQSDVWSFGVLLWEIFSLGASPYPGVQINEEFCQRVRDGTRMRAPELATPAIRHIMLNCWSGDPKARPAFSDLVEILGDLLQGRGLQEEEEVCMAPRSSQSSEEGSFSQVSTMALHIAQADAEDSPPSLQRHSLAARYYNWVSFPGCLARGAETRGSSRMKTFEEFPMTPTTYKGSVDNQTDSGMVLASEEFEQIESRHRQESGFSCKGPGQNVAVTRAHPDSQGRRRRPERGARGGQVFYNSEYGELSEPSEEDHCSPSARVTFFTDNSY

SEQ ID NO: 3 - DNA, 2015 bp, Homo sapiens
  CDS (372)...(1628)
cgcggggtgttctggtgtcccccgccccgcctctccaaaaagctacaccgacgcggaccgcggcggcgtcctccctcgccctcgcttcacctcgcgggctccgaatgcggggagctcggatgtccggtttcctgtgaggcttttacctgacacccgccgcctttccccggcactggctgggagggcgccctgcaaagttgggaacgcggagccccggacccgctcccgccgcctccggctcgcccagggggggtcgccgggaggagcccgggggagagggaccaggaggggcccgcggcctcgcaggggcgcccgcgcccccacccctgcccccgccagcggaccggtcccccacccccggtccttccaccatgcacttgctgggcttcttctctgtggcgtgttctctgctcgccgctgcgctgctcccgggtcctcgcgaggcgcccgccgccgccgccgccttcgagtccggactcgacctctcggacgcggagcccgacgcgggcgaggccacggcttatgcaagcaaagatctggaggagcagttacggtctgtgtccagtgtagatgaactcatgactgtactctacccagaatattggaaaatgtacaagtgtcagctaaggaaaggaggctggcaacataacagagaacaggccaacctcaactcaaggacagaagagactataaaatttgctgcagcacattataatacagagatcttgaaaagtattgataatgagtggagaaagactcaatgcatgccacgggaggtgtgtatagatgtggggaaggagtttggagtcgcgacaaacaccttctttaaacctccatgtgtgtccgtctacagatgtgggggttgctgcaatagtgaggggctgcagtgcatgaacaccagcacgagctacctcagcaagacgttatttgaaattacagtgcctctctctcaaggccccaaaccagtaacaatcagttttgccaatcacacttcctgccgatgcatgtctaaactggatgtttacagacaagttcattccattattagacgttccctgccagcaacactaccacagtgtcaggcagcgaacaagacctgccccaccaattacatgtggaataatcacatctgcagatgcctggctcaggaagattttatgttttcctcggatgctggagatgactcaacagatggattccatgacatctgtggaccaaacaaggagctggatgaagagacctgtcagtgtgtctgcagagcggggcttcggcctgccagctgtggaccccacaaagaactagacagaaactcatgccagtgtgtctgtaaaaacaaactcttccccagccaatgtggggccaaccgagaatttgatgaaaacacatgccagtgtgtatgtaaaagaacctgccccagaaatcaacccctaaatcctggaaaatgtgcctgtgaatgtacagaaagtccacagaaatgcttgttaaaaggaaagaagttccaccaccaaacatgcagctgttacagacggccatgtacgaaccgccagaaggcttgtgagccaggattttcatatagtgaagaagtgtgtcgttgtgtcccttcatattggaaaagaccacaaatgagctaagattgtactgttttccagttcatcgattttctattatggaaaactgtgttgccacagtagaactgtctgtgaacagagagacccttgtgggtccatgctaacaaagacaaaagtctgtctttcctgaaccatgtggataactttacagaaatggactggagctcatctgcaaaaggcctcttgtaaagactggttttctgccaatgaccaaacagccaagattttcctcttgtgatttctttaaaagaatgactatataatttatttccactaaaaatattgtttctgcattcatttttatagcaacaacaattggtaaaactcactgtgatcaatatttttatatcatgcaaaatatgtttaaaataaaatgaaaattgtatt

SEQ ID NO: 4 - protein, 419 aa, Homo sapiens
MHLLGFFSVACSLLAAALLPGPREAPAAAAAFESGLDLSDAEPDAGEATAYASKDLEEQLRSVSSVDELMTVLYPEYWKMYKCQLRKGGWQHNREQANLNSRTEETIKFAAAHYNTEILKSIDNEWRKTQCMPREVCIDVGKEFGVATNTFFKPPCVSVYRCGGCCNSEGLQCMNTSTSYLSKTLFEITVPLSQGPKPVTISFANHTSCRCMSKLDVYRQVHSIIRRSLPATLPQCQAANKTCPTNYMWNNHICRCLAQEDFMFSSDAGDDSTDGFHDICGPNKELDEETCQCVCRAGLRPASCGPHKELDRNSCQCVCKNKLFPSQCGANREFDENTCQCVCKRTCPRNQPLNPGKCACECTESPQKCLLKGKKFHHQTCSCYRRPCTNRQKACEPGFSYSEEVCRCVPSYWKRPQMS

SEQ ID NO: 5 - protein, 15 aa, Artificial Sequence
  synthetic construct
CXXXXXXXXXXCXCC

SEQ ID NO: 6 - DNA, 13 bp, Artificial Sequence
  synthetic construct
nnyngucnnnnnn

SEQ ID NO: 7 - DNA, 4 bp, Artificial Sequence
  synthetic construct
nguc

(19) United States
(12) Patent Application Publication
(10) Pub. No.: US 2003/0180294 A1
(43) Pub. Date: Sep. 25, 2003

(54) Methods of extending corneal graft survival

(76) Inventor: Gerald W. DeVries, Laguna Hills, CA (US)

Correspondence Address: CATHRYN CAMPBELL, CAMPBELL & FLORES LLP, 7th Floor, 4370 La Jolla Village Drive, San Diego, CA, 92122, US

(21) Appl. No.: 10/081,126

(22) Filed: Feb. 22, 2002

(51) Int. Cl. A61K 48/00; A61K 39/395

(52) U.S. Cl. 424/143.1; 514/44

(57) ABSTRACT

The present invention provides a method of extending corneal graft survival following corneal transplantation in a patient by administering to the patient an effective amount of a pharmaceutical composition containing a vascular endothelial growth factor receptor-3 (VEGFR-3) inhibitor, whereby lymphangiogenesis is suppressed in the cornea of the patient.

### BACKGROUND OF THE INVENTION

1. Field of the Invention

The present invention relates generally to the fields of ophthalmology, transplantation and molecular medicine and, in particular, to the use of drugs that regulate lymphangiogenesis for inhibiting corneal allograft rejection.

2. Background Information

Corneal transplantation is, arguably, the most successful tissue transplantation procedure in humans, due in part to the relative immunological privilege of the cornea. The overall first year survival rate of corneal transplants is as high as 90%, even in the absence of routine HLA typing and with minimal immunosuppressive therapy. However, the initial success of corneal transplantation is marred by longer term success rates, which diminish to about 74% by year 5 and about 62% by year 10. Furthermore, in high risk patients such as those with corneal neovascularization or ongoing ocular inflammation, the 10 year graft survival rate is less than 35%. Despite advances in immunological, surgical procedures and medical management, corneal graft survival has not improved over the last ten years (Naacke et al., Cornea 350-353 (2001); Waldock and Cook, Brit. J. Ophthal. 84:813-815 (2000); and Foulks, “Clinical Aspects of Corneal Allograft Rejection,” in Krachmer et al., Cornea Volume III pages 1687-1696 (1997)). In addition, because corneal transplantation is relatively common with about 45,000 surgeries performed per year in the United States, allograft rejection effects a large number of individuals.

The primary cause of corneal transplant failure is allograft rejection. Unfortunately, current treatments for allograft rejection, principally immunosuppressive agents such as corticosteroids, are effective in only about 50% of cases. Furthermore, in spite of evidence that recipient corneal vascularization is associated with graft failure, inhibition of allograft vascularization, for example, with a platelet-activating factor (PAF) antagonist, has not been successful in increasing graft survival (Cohen et al., Curr. Eye Res. 13:139-144 (1994)). Thus, there is a need for novel methods of treating corneal allograft rejection to extend graft survival. The present invention satisfies this need and provides related advantages as well.

### SUMMARY OF THE INVENTION

The present invention provides a method of extending corneal graft survival following corneal transplantation in a patient by administering to the patient an effective amount of a pharmaceutical composition containing a vascular endothelial growth factor receptor-3 (VEGFR-3) inhibitor, whereby lymphangiogenesis is suppressed in the cornea of the patient.

In one embodiment, the present invention provides a method of extending corneal graft survival following corneal transplantation in a patient by administering to the patient an effective amount of a pharmaceutical composition containing a dominant negative VEGFR-3 receptor, whereby lymphangiogenesis is suppressed in the cornea of the patient. Such a dominant negative VEGFR-3 receptor can be, for example, a kinase-inactive VEGFR-3 receptor or a soluble VEGFR-3 receptor. Similarly, a VEGFR-3 inhibitor useful for extending corneal graft survival can be, for example, a nucleic acid molecule encoding a dominant negative VEGFR-3 receptor such as a kinase-inactive VEGFR-3 receptor or a soluble VEGFR-3 receptor.

The present invention also provides a method of extending corneal graft survival following corneal transplantation in a patient by administering to the patient an effective amount of a pharmaceutical composition containing a VEGFR-3 kinase inhibitor, whereby lymphangiogenesis is suppressed in the cornea of the patient. In one embodiment, the VEGFR-3 kinase inhibitor binds the VEGFR-3 catalytic domain, and, in another embodiment, the VEGFR-3 kinase inhibitor is an ATP analog.

The present invention additionally provides a method of extending corneal graft survival following corneal transplantation in a patient by administering to the patient an effective amount of a pharmaceutical composition containing a VEGFR-3 inhibitor that is a VEGFR-3 binding molecule, whereby lymphangiogenesis is suppressed in the cornea of the patient. Such a VEGFR-3 binding molecule can bind, for example, the extracellular domain of VEGFR-3. A VEGFR-3 binding molecule useful in the invention also can be anti-VEGFR-3 antibody material, which, in one embodiment, is monoclonal antibody material.

Further provided by the invention is a method of extending corneal graft survival following corneal transplantation in a patient by administering to the patient an effective amount of a pharmaceutical composition containing a VEGFR-3 inhibitor that down-regulates VEGFR-3 expression, whereby lymphangiogenesis is suppressed in the cornea of the patient. Such a VEGFR-3 inhibitor can be, for example, a sequence-specific ribonuclease such as a ribozyme or a VEGFR-3 antisense nucleic acid molecule.

The invention also provides a method of extending corneal graft survival following corneal transplantation in a patient by administering to the patient an effective amount of a pharmaceutical composition containing anti-VEGF-C neutralizing antibody material, whereby lymphangiogenesis is suppressed in the cornea of the patient. Anti-VEGF-C neutralizing antibody material useful in the invention can be, for example, monoclonal antibody material.

In addition, the invention provides a method of extending corneal graft survival following corneal transplantation in a patient by administering to the patient an effective amount of a pharmaceutical composition containing a VEGFR-3 inhibitor that down-regulates VEGF-C expression, whereby lymphangiogenesis is suppressed in the cornea of the patient. Such a VEGFR-3 inhibitor can be, for example, a sequence-specific ribonuclease such as a ribozyme, or can be, for example, a VEGF-C antisense nucleic acid molecule.

The invention also provides a method of extending corneal graft survival following corneal transplantation in a patient by administering to the patient an effective amount of a pharmaceutical composition containing a cell that expresses a VEGFR-3 inhibitor, whereby lymphangiogenesis is suppressed in the cornea of the patient.

In a method of the invention, an anti-angiogenic agent can be administered to the patient in addition to the pharmaceutical composition containing the VEGFR-3 inhibitor. Similarly, an immunosuppressive agent can be administered to the patient in addition to the pharmaceutical composition containing the VEGFR-3 inhibitor and, if desired, can be administered in conjunction with an anti-angiogenic agent.

In the methods of the invention, a pharmaceutical composition containing a VEGFR-3 inhibitor can be administered prior to, during, or subsequent to corneal transplantation. Furthermore, administration of the pharmaceutical composition containing VEGFR-3 inhibitor can be repeated, as needed. In one embodiment, administration is repeated over a period of at least one month. In another embodiment, administration is repeated over a period of at least six months.

Also provided by the invention is a method of extending corneal graft survival following corneal transplantation in a patient by administering to the patient prior to corneal transplantation an effective amount of a pharmaceutical composition containing a VEGFR-3 inhibitor; and administering to the patient subsequent to corneal transplantation an effective amount of a pharmaceutical composition containing a VEGFR-3 inhibitor, whereby lymphangiogenesis is suppressed in the cornea of the patient. The pre- and post-surgical pharmaceutical compositions can be the same or different and can be administered using the same or different routes of delivery.

A variety of routes of administration can be useful in the methods of the invention. In one embodiment, a method of the invention for extending corneal graft survival is practiced by systemic administration of the pharmaceutical composition. In another embodiment, a method of the invention is practiced by local administration of the pharmaceutical composition. In further embodiments, the pharmaceutical composition is administered topically, or by local injection, or is released from an intraocular or periocular implant.

### BRIEF DESCRIPTION OF THE DRAWINGS

FIG. 1 shows the structure of endothelial-cell receptor tyrosine kinases and growth factors involved in vasculogenesis, angiogenesis and lymphangiogenesis. The structurally divergent Tie and vascular endothelial growth factor (VEGF) receptor families are shown with the specificity of ligand binding to the receptors is indicated by arrows. The VEGF receptor family contains three transmembrane receptors, VEGFR-1, VEGFR-2 and VEGFR-3. A soluble form of VEGFR-1 (sVEGFR-1) has also been characterized. The extracellular regions of the VEGF receptors contain seven immunoglobulin domains that are stabilized by disulfide links (SS) between paired cysteine residues; in VEGFR-3, the fifth domain is proteolytically processed into two disulfide-linked polypeptides. In the intracellular region of the VEGF receptors, the tyrosine kinase domains are interrupted by a small stretch of amino acids commonly referred to as a kinase insert. Some biological processes mediated by the receptors also are indicated.

FIG. 2 shows the nucleotide and amino acid sequence of human vascular endothelial growth factor receptor-3 (VEGFR-3). A. The nucleotide sequence (SEQ ID NO: 1) of human VEGFR-3. B. The amino acid sequence (SEQ ID NO: 2) of human VEGFR-3. The start codon is underlined. Genbank accessions X69878 and S66407. See, also, Galland et al., Oncogene 8:1233-1240 (1993) and Pajusola et al., Oncogene 8:2931-2937 (1993).

FIG. 3 shows the nucleotide and amino acid sequence of human vascular endothelial growth factor-C (VEGF-C). A. The nucleotide sequence (SEQ ID NO: 3) of human VEGF-C. B. The amino acid sequence (SEQ ID NO: 4) of human VEGF-C. The start codon is underlined. Genbank accession NM_{—}005429.

### DETAILED DESCRIPTION OF THE INVENTION

The present invention provides a method of extending corneal graft survival following corneal transplantation in a patient by administering to the patient an effective amount of a pharmaceutical composition containing a vascular endothelial growth factor receptor-3 (VEGFR-3) inhibitor, whereby lymphangiogenesis is suppressed in the cornea of the patient.

The methods of the invention are useful to extend corneal graft survival following corneal transplantation in a patient. As used herein, the term “corneal transplantation” refers to any procedure whereby allogeneic or xenogeneic corneal tissue is orthotopically grafted to a recipient patient. In one embodiment, allogeneic corneal tissue is grafted in the corneal transplantation procedure. In a further embodiment, the corneal transplantation procedure is a penetrating keratoplasty, in which a section of full-thickness cornea is transplanted. The methods of the invention also are applicable to corneal transplantation procedures such as lamellar keratoplasty, in which the anterior half of the cornea is transplanted with the anterior chamber remaining intact; optic keratoplasty, in which donor corneal material is transplanted to replace recipient scar tissue that interferes with vision; refractive keratoplasty, in which a section of donor cornea is shaped to the desired curvature, and inserted between layers of recipient cornea, or on recipient's cornea, to change the recipient's corneal curvature and correct optical errors; and tectonic keratoplasty, in which corneal material is transplanted to replace lost recipient tissue, for example, following trauma.

HLA class I antigens are expressed in abundance on corneal epithelial, stromal, and endothelial cells, while there is relatively low indigenous expression of MHC class II molecules within the cornea, either on Langerhans cells in the epithelium or dendritic cells present within the stroma (Treseler, Am. J. Ophthalmol. 98:763-772 (1984); McCallum et al., Invest. Ophthalmol. Vis. Sci. 34:1793-1803 (1993)). It is understood that the methods of the invention can be useful to extend corneal graft survival following the transplantation of a corneal graft that has been matched to the recipient patient for one or more HLA antigens (Waldock and Cook, supra, 2000). Such a molecule can be a major or class I antigens (HLA-A and HLA-B) or a minor or class II antigen (HLA-DR).

Thus, a method of the invention can be practiced to extend survival of a corneal graft that has been selected, for example, to share at least one HLA class I antigen, or at least two HLA class I antigens, with the recipient patient. Similarly, a method of the invention can be practiced to extend survival of a corneal graft that has been selected to share at least one HLA class II antigen with the recipient patient, or that has been selected to share at least one HLA class I antigen and at least one HLA class II antigen with the recipient patient. A method of the invention also can be practiced, for example, to extend survival of a corneal graft that has been selected to share at least one HLA class I antigen but which is mismatched for HLA class II antigens.

The term “patient,” as used herein, means the recipient of donor corneal tissue in a corneal transplantation procedure. A patient can be, for example, a mammal such as a primate, rabbit or rodent. In one embodiment, the patient is a human patient.

The methods of the invention are practiced to extend corneal graft survival following corneal transplantation. As used herein, the phrase “extend corneal graft survival” means that, on average, irreversible graft rejection is delayed or prevented. Thus, corneal graft survival is “extended” in a population when the number of months prior to irreversible allograft rejection is increased, on average, in the population, as compared to a corresponding population that was not treated with a pharmaceutical composition containing a VEGFR-3 inhibitor. Corneal graft survival also is extended in a population when the percentage of individuals with irreversible graft rejection decreases, on average, in the population, as compared to a corresponding population that was not treated with a pharmaceutical composition containing a VEGFR-3 inhibitor.

One skilled in the art uses established criteria to determine whether there is irreversible graft rejection. Rejection generally is evidenced as one or more pathologic events that involve the grafted cornea and progress toward the center of the graft but which do not effect the recipient cornea. Epithelial rejection is characterized by an epithelial rejection line appearing as a raised ridge of epithelium; subepithelial rejection is characterized by subepithelial infiltrates that resemble those seen in epidemic keratoconjunctivitis. Furthermore, stromal rejection is characterized by stromal infiltrates that progress toward the center of the graft, and endothelial rejection is characterized by at least one of the following: a Khodadoust line, keratic precipitates, stromal edema or aqueous cells. One skilled in the art understands that, in many cases, rejection is reversible with treatment such as topical dexamethasone; topical dexamethasone accompanied by subconjunctival dexamethasone injection and, if needed, accompanied by intravenous methylprednisone for several days. Rejection is considered irreversible when signs of rejection (rejection lines, subepithelial infiltrates, keratic precipitates, stromal infiltrates, stromal edema and aqueous cells) observed using slit-lamp examination fail to disappear; or there is abnormal graft thickness or loss of visual acuity.

The methods of the invention rely on an inhibitor of vascular endothelial growth factor receptor-3 or another anti-lymphangiogenic agent. There are at least three vascular endothelial growth factor receptors: VEGFR-1, VEGFR-2 and VEGFR-3, originally named Flt1 (Fms-like tyrosine kinase, KDR/Flk-1 (kinase insert-domain containing receptor or fetal-liver kinase) and Flt4, respectively. These subclass-III receptor tyrosine kinases, which are homologous to the platelet-derived growth factor (PDGF)-receptor family, are characterized by seven immunoglobulin homology domains in the extracellular domain, and a tyrosine kinase intracellular domain split by a kinase insert sequence (Klagsbrun and D'Amore, Cytokine Growth Factor Rev. 7:259-270 (1996)).

Human VEGFR-3 shows approximately 35% amino acid identity with VEGFR-1 and VEGFR-2 in the extracellular domain and about 80% in the tyrosine kinase domain. Human VEGFR-3 has been cloned from placental and erythroleukemia cell cDNA libraries (Aprelikova et al., Cancer Res. 52:746-748 (1992); Galland et al., Genomics 13:475-4878 (1992); Galland et al., supra, 1993; Pajusola et al., Cancer Res. 52:5738-5743 (1992); and Pajusola et al., supra, 1993, and mouse and quail homologs also have been cloned (Finnerty et al., Oncogene 8:2293-2298 (1993); Eichmann et al., Gene 174:3-8 (1996)). VEGFR-3 homologs are well conserved in evolution, with the quail homolog having about 70% amino acid identity with the human receptor and similar ligand-binding characteristics.

The major human VEGFR-3 mRNA transcript is about 5.8 kb in size; an alternative 3′ polyadenylation signal results in a minor 4.5 kb transcript encoding a protein with a 65 residue truncation at the C-terminus. The longer form of VEGFR-3, which is the major form detected in tissues, is synthesized as a 195 kDa precursor that is glycosylated and proteolytically cleaved after Arg472 to yield a disulfide linked two-chain form. In the carboxy-terminal region of the longer form are three tyrosine residues not encoded in the shorter transcript: Tyr 1333, Tyr 1337 and Tyr 1363.

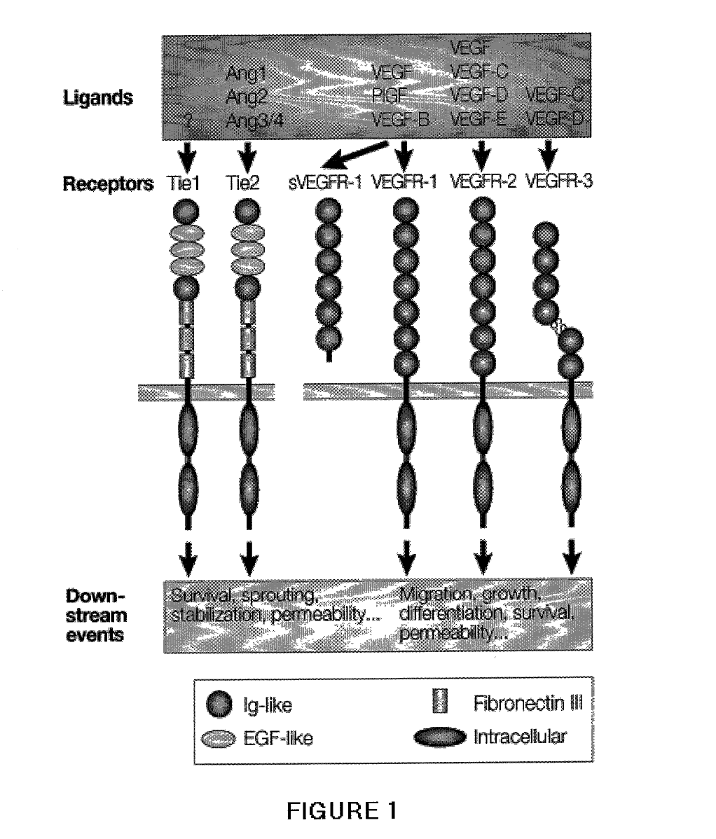

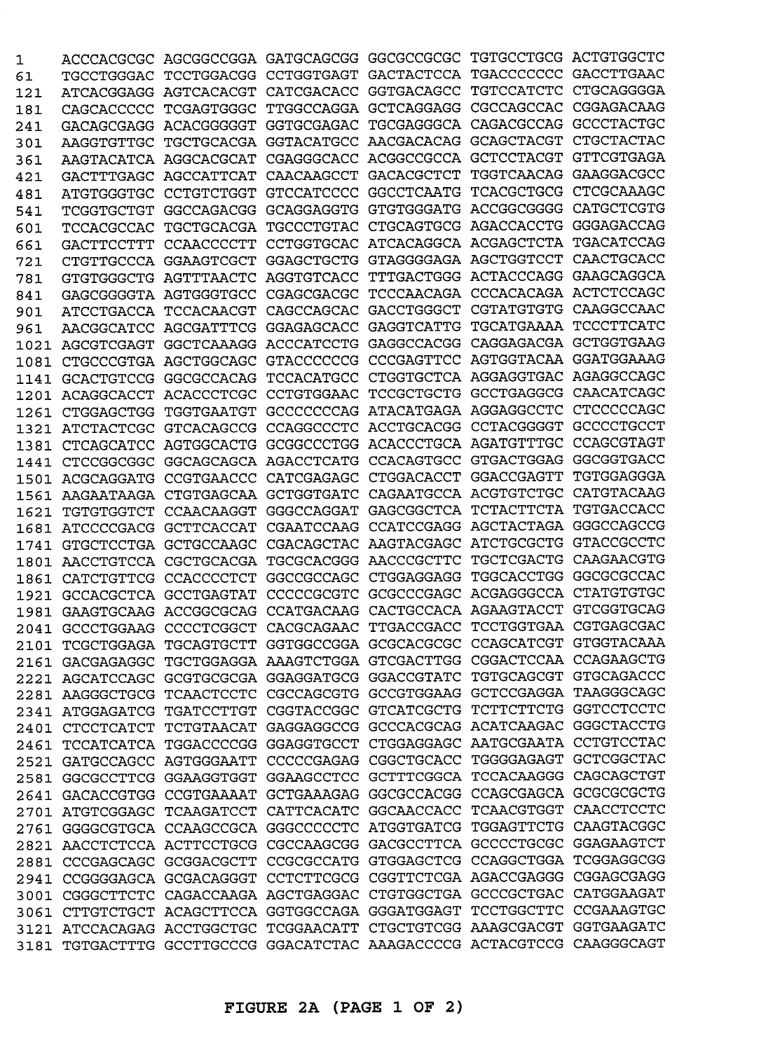

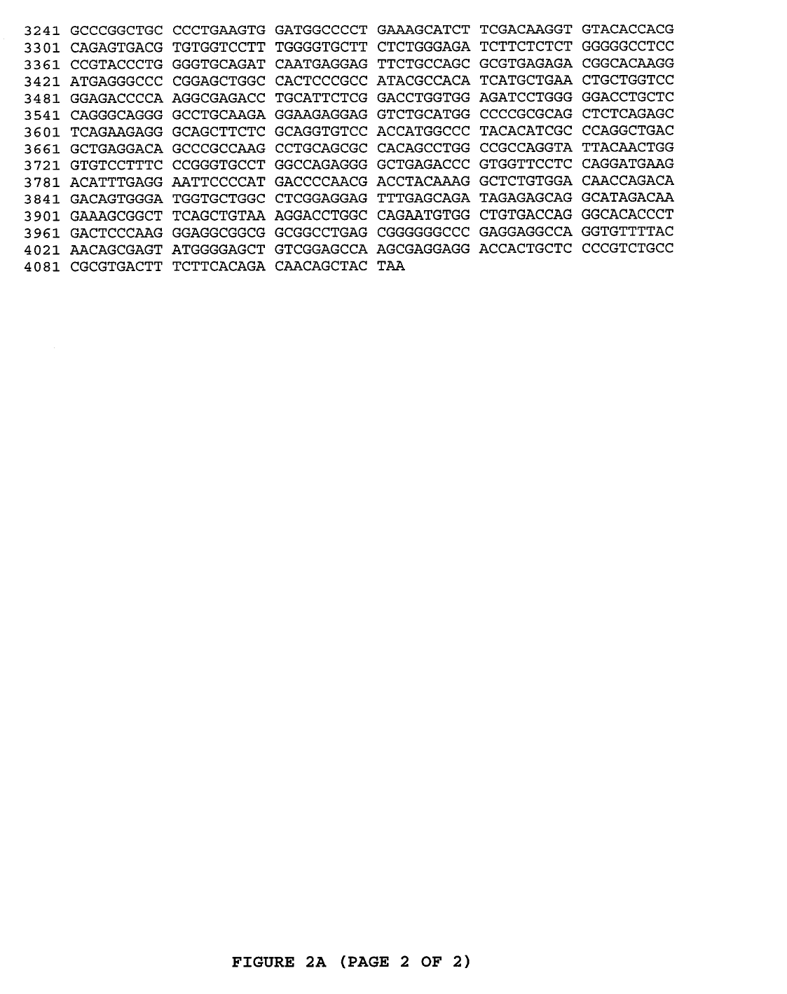

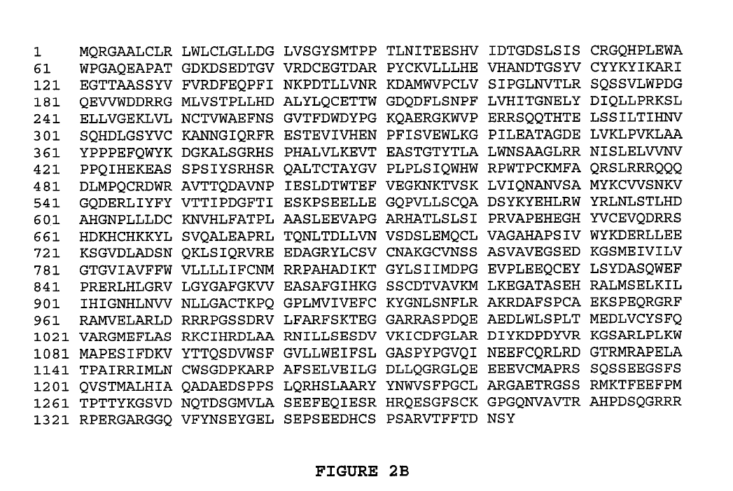

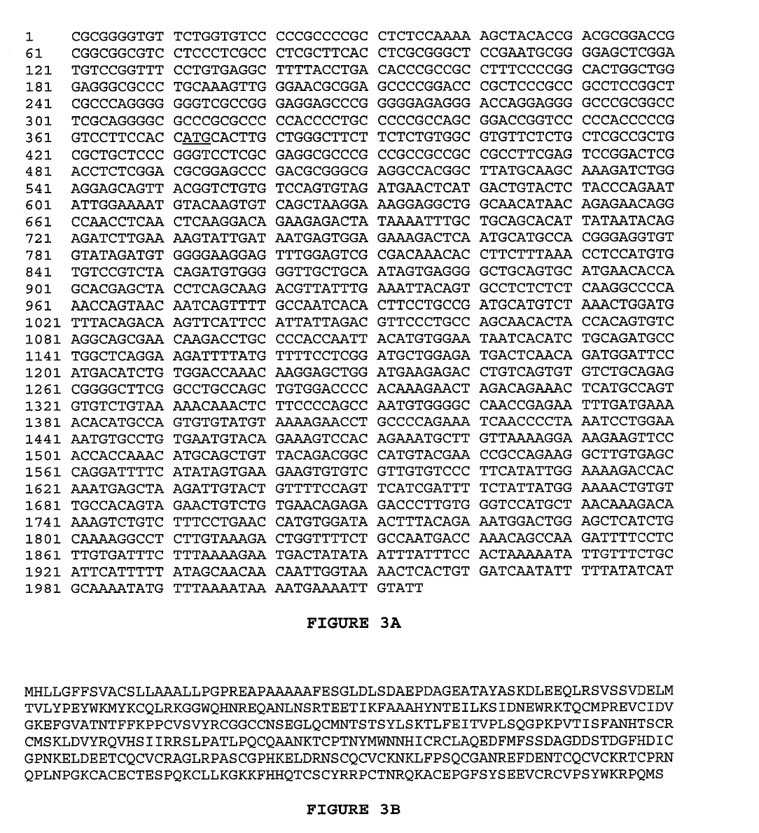

VEGFR-3 has an amino-terminal extracellular domain, a small transmembrane region and a carboxy-terminal cytoplasmic domain. The extracellular domain of VEGFR-3 has seven immunoglobulin-like C2-type domains; upon dimerization, the protein becomes disulfide bonded within the fifth immunoglobulin-like domain. VEGFR-3 is a type I membrane protein containing a transmembrane region of about 20 residues; the carboxy-terminal cytoplasmic domain includes two tyrosine kinase domains (see FIG. 1). As shown in FIG. 2B, the long isoform of human VEGFR-3 (SEQ ID NO: 2) is a protein of 1363 residues, with amino acids 24 to 1363 making up the mature protein. Residues 24 to 775 of human VEGFR-3 (SEQ ID NO: 2) make up the extracellular domain; residues 776 to 797 of SEQ ID NO: 2 make up the transmembrane region; and residues 798 to 1363 of SEQ ID NO: 2 make up the cytoplasmic domain. The seven immunoglobulin-like domains can be localized within the extracellular portion of human VEGFR-3 (SEQ ID NO: 2) as follows: immunoglobulin-like domain 1 (residues 44 to 118); immunoglobulin-like domain 2 (residues 151 to 213); immunoglobulin-like domain 3 (residues 245 to 317); immunoglobulin-like domain 4 (residues 351 to 403); immunoglobulin-like domain 5 (residues 438 to 541); immunoglobulin-like domain 6 (residues 571 to 660); and immunoglobulin-like domain 7 (residues 692 to 758). The ligand-binding domain of VEGFR is made up of the first three immunoglobulin-like domains.

The vascular endothelial growth factors, VEGF-A, VEGF-B, VEGF-C, and VEGF-D, share structural features typical but display different biological activities attributable to different specificities for VEGF receptors, VEGFR-1, VEGFR-2 and VEGFR-3. Within the VEGF family of growth factors, VEGF-C and VEGF-D are most closely related and form a subgroup characterized by unique amino- and carboxy-terminal extensions flanking the common VEGF-homology domain. Human VEGF-C is a protein of 419 amino acids with a predicted molecular mass of 46.9 kDa; murine VEGF-C is a protein of 415 amino acids.

The central core (VEGF homology domain) exhibits about 30% amino acid identity to VEGF and is encoded by the third and fourth of seven exons, as for other members of the VEGF family. The VEGF homology domains of VEGF-C and VEGF-D share 60% amino acid identity. The carboxy-terminal domain contains a repetitive pattern of cysteine residues, Cys-X₁₀-Cys-X-Cys-Cys (SEQ ID NO: 5), similar to a motif present in the Balbiani ring 3 protein, a secretory protein which is a component of silk produced in larval salivary glands of the midge Chironomus tentans.

VEGF-C is synthesized as a precursor, subsequently proteolytically processed in a manner similar to PDGF-A and B chain processing. VEGF-C is secreted as a disulfide-bonded homodimer containing the C-terminal silk domain. Following secretion, the carboxy-terminal silk domain is cleaved and disulfide bonded to the amino-terminal domain to produce a disulfide-linked tetramer composed of 29 and 31 kDa polypeptides. Proteolytic processing of the amino-terminal propeptide releases the mature form made up of two 21 kDa polypeptide chains encoding the VEGF homology domain.

As disclosed herein, corneal graft survival can be extended by treatment of the patient by a VEGFR-3 inhibitor. As used herein, the term “VEGFR-3 inhibitor” means a molecule that reduces VEGFR-3 expression, activity or intracellular signaling. Such an inhibitor can be, for example, a small molecule, protein, peptide, peptidomimetic, ribozyme, nucleic acid molecule or oligonucleotide, oligosaccharide, cell, phage or virus, or a combination thereof. As described further below, VEGFR-3 inhibitors useful in the invention encompass, without limitation, dominant negative VEGFR-3 receptors including soluble receptors and kinase inactive receptors; VEGFR-3 kinase inhibitors, including selective VEGFR-3 kinase inhibitors and molecules that bind the VEGFR-3 catalytic domain such as ATP analogs; VEGFR-3 binding molecules including molecules that bind the VEGFR-3 extracellular domain, including antibodies, proteins, small molecules and oligonucleotides that prevent or diminish ligand binding to VEGFR-3; anti-VEGF-C antibodies; VEGF-C antagonists; conjugates in which a VEGFR-3 ligand is linked to a toxin; ribozymes, antisense nucleic acid molecules and nucleic acid molecules encoding negative regulatory transcription factors that prevent or reduce VEGFR-3 expression, as well as cells or viruses containing such ribozymes and nucleic acid molecules; ribozymes, antisense nucleic acid molecules and nucleic acid molecules encoding negative regulatory transcription factors that prevent or reduce VEGF-C expression, and cells and viruses containing such ribozymes or nucleic acid molecules; nucleic acid molecules encoding, for example, dominant negative VEGFR-3 receptors, transcription factors, and antibodies and antigen-binding fragments thereof, and cells and viruses including such nucleic acid molecules; and selective inhibitors of VEGFR-3 intracellular signaling. One skilled in the art understands that these and other VEGFR-3 inhibitors can be useful in the methods of the invention, as described further below.

A VEGFR-3 inhibitor can be a specific, selective or non-selective inhibitor of VEGFR-3 expression, activity or intracellular signaling. A specific VEGFR-3 inhibitor reduces the expression, activity or intracellular signaling of VEGFR-3 in preference to the activity of most or all unrelated receptor tyrosine kinases such as FGFR1 and in preference to the activity of VEGFR-1 and VEGFR-2. A selective VEGFR-3 inhibitor reduces the expression, activity or intracellular signaling of VEGFR-3 in preference to most or all unrelated receptor tyrosine kinases such as FGFR1. In contrast, a non-selective VEGFR-3 inhibitor reduces the expression, activity or intracellular signaling of VEGFR-1 or VEGFR-2 or both to a similar extent as VEGFR-3. One skilled in the art recognizes that specific, selective and non-selective VEGFR-3 kinase inhibitors can be useful in the methods disclosed herein.

As set forth herein, a variety of VEGFR-3 inhibitors are useful for extending corneal graft survival according to a method of the invention. In one embodiment, the invention provides a method of extending corneal graft survival following corneal transplantation in a patient by administering to the patient an effective amount of a pharmaceutical composition containing a dominant negative VEGFR-3 receptor, whereby lymphangiogenesis is suppressed in the cornea of the patient. Such a dominant negative VEGFR-3 receptor can be, for example, a kinase-inactive VEGFR-3 receptor or a soluble VEGFR-3 receptor. Similarly, a VEGFR-3 inhibitor useful for extending corneal graft survival can be, for example, a nucleic acid molecule encoding a dominant negative VEGFR-3 receptor. In such a method, the nucleic acid molecule can encode, for example, a kinase-inactive VEGFR-3 receptor or a soluble VEGFR-3 receptor.

As used herein, the term “dominant negative VEGFR-3 receptor” means a variant of a wild type VEGFR-3 receptor that acts to reduce activity of wild type VEGFR-3 receptor. While it is recognized that a dominant negative receptor can function through a variety of mechanisms, exemplary mechanisms through which a VEGFR-3 dominant negative receptor can function include, without limitation, depletion of free ligand and formation of inactive wild type/dominant negative receptor dimers. Thus, a dominant negative VEGFR-3 receptor can be a soluble or membrane-bound form of the VEGFR-3 receptor and can include, for example, one or a few point mutations, or a gross deletion of several hundred amino acids relative to the wild type receptor sequence. Exemplary dominant negative VEGFR-3 receptors include, without limitation, a variant VEGFR-3 receptor consisting essentially of the cytoplasmic domain (soluble VEGFR-3) or another soluble receptor containing a functional ligand-binding domain; a variant VEGFR-3 receptor consisting essentially of the cytoplasmic and transmembrane domains; a variant VEGFR-3 receptor with an inactive tyrosine kinase domain having, for example, a deletion of some or all of the tyrosine kinase domain or one or more point substitutions within the tyrosine kinase domain. It is understood that a dominant negative VEGFR-3 receptor also can contain one or more heterologous sequences in addition to the VEGFR-3 receptor sequence. Methods for preparing dominant negative vascular endothelial growth factor receptors are well known in the art. See, for example, Mäkinen et al., Nature Medicine 7:199-205 (2001); and Millauer et al., Nature 367:576-579 (1994).

A dominant negative VEGFR-3 receptor, or nucleic acid molecule encoding same, acts to reduce activity of endogenous VEGFR-3 receptor present in the patient undergoing corneal transplantation. Where the patient is a human, the dominant negative VEGFR-3 receptor or encoding nucleic acid molecule acts to reduce activity of endogenous human VEGFR-3 receptor. In the human VEGFR-3 receptor (long isoform) shown in FIG. 2B, residues 24 to 775 of SEQ ID NO: 2 make up the extracellular domain; residues 776 to 797 of SEQ ID NO: 2 make up the transmembrane domain; and residues 798 to 1363 of SEQ ID NO: 2 make up the cytoplasmic domain, with the tyrosine kinase domain positioned from amino acids 845 to 1173. The short isoform is similar to the long isoform, but lacks the carboxy-terminal 65 residues. Exemplary dominant negative human VEGFR-3 receptors include, without limitation, soluble human VEGFR-3 receptor variants such as the variant having residues 24 to 350 of SEQ ID NO: 2 (ligand-binding domain containing immunoglobulin-like domains 1 to 3) or the variant having residues 24 to 775 (complete extracellular domain), or nucleic acid molecules encoding these variants; the human VEGFR-3 receptor variant having residues 24 to 797 (extracellular and transmembrane domains), or a nucleic acid molecule encoding this variant; the human VEGFR-3 receptor variant having residues 24 to 844 (deleted for tyrosine kinase domain), or a nucleic acid molecule encoding this variant.

In one embodiment, the invention provides a method of extending corneal graft survival following corneal transplantation in a patient by administering a VEGFR-3 inhibitor which is a soluble VEGFR-3 receptor. Such a soluble VEGFR-3 receptor lacks a functional transmembrane domain. A soluble VEGFR-3 receptor can be a VEGFR-3 variant with a deletion of the native transmembrane domain. In one embodiment, a soluble VEGFR-3 receptor consists of the extracellular domain or a portion thereof. Such a soluble VEGFR-3 receptor can be a VEGFR-3 variant having, for example, three, four, five, six or seven of the extracellular Ig-homology domains of a VEGFR-3 such as human VEGFR-3. This and other soluble VEGFR-3 receptors can be prepared by routine methods. See, for example, Mäkinen et al., supra, 2001, which describes a soluble VEGFR-3 receptor consisting of the three amino-terminal Ig-homology domains of VEGFR-3 and an IgG Fc domain, which binds VEGF-C with the same efficiency as the full-length extracellular domain and inhibits VEGF-C-induced VEGFR-3 phosphorylation and subsequent p42/p44 mitogen-activated protein kinase (MAPK) activation in VEGFR-3 expressing endothelial cells.

The invention also provides a method of extending corneal graft survival following corneal transplantation in a patient by administering to the patient an effective amount of a pharmaceutical composition containing a VEGFR-3 kinase inhibitor, whereby lymphangiogenesis is suppressed in the cornea of the patient. In one, the VEGFR-3 kinase inhibitor binds the VEGFR-3 catalytic domain, and, in a further embodiment, the VEGFR-3 kinase inhibitor is an ATP analog.

As used herein, the term “VEGFR-3 kinase inhibitor” means an inhibitor of receptor tyrosine kinase activity that selectively or non-selectively reduces the tyrosine kinase activity of a VEGFR-3 receptor. Such an inhibitor generally reduces VEGFR-3 tyrosine kinase activity without significantly effecting the expression of VEGFR-3 and without effecting other VEGFR-3 activities such as ligand-binding capacity. A VEGFR-3 kinase inhibitor can be a molecule that directly binds the VEGFR-3 catalytic domain, for example, an ATP analog. A VEGFR-3 kinase inhibitor can bind the VEGFR-3 catalytic domain through one or more hydrogen bonds similar to those anchoring the adenine moiety of ATP to VEGFR-3 (Engh et al., J. Biol. Chem. 271:26157-26164 (1996); Tong et al., Nature Struc. Biol. 4:311-316 (1997); and Wilson et al., Chem. Biol. 4:423-431 (1997)). A VEGFR-3 kinase inhibitor also can bind the hydrophobic pocket adjacent to the adenine binding site (Mohamedi et al., EMBO J. 17:5896-5904 (1998); Tong et al., supra, 1997; and Wilson et al., supra, 1997).

VEGFR-3 kinase inhibitors useful in the invention include specific VEGFR-3 kinase inhibitors such as indolinones that differentially block VEGF-C and VEGF-D induced VEGFR-3 kinase activity compared to that of VEGFR-2. Such specific VEGFR-3 kinase inhibitors, for example, MAE106 and MAZ51 can be prepared as described in Kirkin et al., Eur. J. Biochem. 268:5530-5540 (2001). Additional VEGFR-3 kinase inhibitors, including specific, selective and non-selective inhibitors, are known in the art or can be identified using one of a number of well known methods for assaying for receptor tyrosine kinase inhibition.

As an example, a VEGFR-3 kinase inhibitor can be identified using a well known ELISA assay to analyze production of phosphorylated tyrosine as described, for example in Hennequin et al., J. Med. Chem. 42:5369-5389 (1999) and Wedge et al., Cancer Res. 60:970-975 (2000). Such an assay can be used to screen for molecules that inhibit VEGFR-3 in preference to other vascular endothelial growth factor receptors such as VEGFR-1 and in preference to unrelated tyrosine kinases such as fibroblast growth factor receptor1 (FGFR1). Briefly, molecules to be screened can be incubated for 20 minutes at room temperature with a cytoplasmic receptor domain in a HEPES (pH 7.5) buffered solution containing 10 mM MnCl₂ and 2 μM ATP in 96-well plates coated with a poly(Glu, Ala, Tyr) 6:3:1 random copolymer substrate (SIGMA; St. Louis, Mo.). Phosphorylated tyrosine can be detected by sequential incubation with mouse IgG anti-phosphotyrosine antibody (Upstate Biotechnology; Lake Placid, N.Y.), a horseradish peroxidase-linked sheep anti-mouse immunoglobulin antibody (Amersham; Piscataway, N.J.), and 2,2′azino-bis (3-ethylbenzthiazoline-6-sulfonic acid) (Roche Molecular Biochemicals, Indianapolis, Ind.). In such an in vitro kinase assay, the source of VEGFR-3 can be, for example, a lysate prepared from an insect cell infected with recombinant baculovirus containing a cytoplasmic receptor domain, for example, encoding residues 798 to 1363 of human VEGFR-3 (SEQ ID NO: 2).

The term VEGFR-3 kinase inhibitor, as used herein, encompasses specific, selective and non-selective inhibitors of VEGFR-3. A specific VEGFR-3 kinase inhibitor reduces the tyrosine kinase activity of VEGFR-3 in preference to the activity of most or all unrelated receptor tyrosine kinases such as FGFR1 and in preference to the activity of the vascular endothelial growth factor receptors, VEGFR-1 and VEGFR-2. A selective VEGFR-3 kinase inhibitor reduces the tyrosine kinase activity of VEGFR-3 in preference to most or all unrelated receptor tyrosine kinases such as FGFR1. Such a selective VEGFR-3 inhibitor can have an IC₅₀ for inhibition of an isolated VEGFR-3 cytoplasmic domain that is, for example, at least 10-fold less than the IC₅₀ for both VEGFR-1 and VEGFR-2. In particular embodiments, the invention provides a selective VEGFR-3 kinase inhibitor having an IC₅₀ for inhibition of an isolated VEGFR-3 cytoplasmic domain that is at least 20-fold, 30-fold, 40-fold, 50-fold, 100-fold, 200-fold, 300-fold, 400-fold or 500-fold less than the IC₅₀ for both VEGFR-1 and VEGFR-2. In contrast, a non-selective VEGFR-3 kinase inhibitor reduces the tyrosine kinase activity of VEGFR-1 or VEGFR-2 or both to a similar extent as VEGFR-3. It is understood that specific, selective and non-selective VEGFR-3 kinase inhibitors can be useful for extending corneal graft survival according to a method of the invention.

The invention also provides a method of extending corneal graft survival following corneal transplantation in a patient by administering to the patient an effective amount of a pharmaceutical composition containing a VEGFR-3 inhibitor that is a VEGFR-3 binding molecule, whereby lymphangiogenesis is suppressed in the cornea of the patient. Such a VEGFR-3 binding molecule can bind, for example, the extracellular domain of VEGFR-3 or the kinase domain of VEGFR-3. A VEGFR-3 binding molecule useful in the invention also can be anti-VEGFR-3 antibody material, which, in one embodiment, is monoclonal antibody material.

In one embodiment, the anti-VEGFR-3 antibody material binds the ligand-binding site of VEGFR-3 and inhibits binding of VEGF-C or VEGF-D or both to VEGFR-3. Such antibody material can be monoclonal or polyclonal. For example, the anti-mouse VEGFR-3 monoclonal antibody AFL4 blocks binding of VEGF-C to VEGFR-3 and further inhibits receptor signaling (Kubo et al., Blood 96:546-553 (2000)). Anti-VEGFR-3 antibody material useful in the invention can have, for example, an IC₅₀ for inhibition of VEGF-C binding to VEGFR-3 of less than 50 μg/ml, less than 5 μg/ml, less than 0.5 μg/ml, less than 0.05 μg/ml, less than 0.005 μg/ml or less than 0.0005 μg/ml. In particular embodiments, a method of the invention utilizes anti-human-VEGFR-3 antibody material having an IC₅₀ for inhibition of VEGF-C binding to human VEGFR-3 of less than 50 μg/ml, less than 5 μg/ml, less than 0.5 μg/ml, less than 0.05 μg/ml, less than 0.005 μg/ml or less than 0.0005 μg/ml. Anti-VEGFR-3 antibody material which inhibits binding of VEGF-C or VEGF-D or both to VEGFR-3 also can reduce receptor signaling as evidenced, for example, by a reduction in VEGF-C induced tyrosine phosphorylation of VEGFR

In another embodiment, the invention provides a method of extending corneal graft survival following corneal transplantation in a patient, in which an effective amount of a pharmaceutical composition containing anti-VEGF-C neutralizing antibody material is administered to the patient, whereby lymphangiogenesis is suppressed in the patient's cornea. Anti-VEGF-C neutralizing antibody material useful in the invention can be, for example, monoclonal anti-VEGF-C neutralizing antibody material.

As used herein, the term “antibody material” is used in its broadest sense to include polyclonal and monoclonal antibodies, as well as polypeptide fragments of antibodies that retain binding activity for VEGFR-3 or VEGF-C of at least about 1×10⁵ M⁻¹. One skilled in the art understands that anti-VEGFR-3 antibody fragments and anti-VEGF-C antibody fragments, such as Fab, F(ab')₂ and Fv fragments, can retain binding activity for VEGFR-3 or VEGF-C and, thus, are included within the definition of antibody material. In addition, the term “antibody material,” as used herein, encompasses non-naturally occurring antibodies and fragments containing, at a minimum, one V_{H} and one V_{L} domain, such as chimeric antibodies, humanized antibodies and single chain Fv fragments (scFv) that specifically bind VEGFR-3 or VEGF-C. Such non-naturally occurring antibodies can be constructed using solid phase peptide synthesis, produced recombinantly or obtained, for example, by screening combinatorial libraries consisting of variable heavy chains and variable light chains as described by Borrebaeck (Ed.), Antibody Engineering (Second edition) New York: Oxford University Press (1995)).

Antibody material “specific for” VEGFR-3, or that “specifically binds” VEGFR-3, binds with substantially higher affinity to VEGFR-3 than to most or all unrelated receptor tyrosine kinases such as FGFR1 and other vascular endothelial growth factor receptors such as VEGFR-1 and VEGFR-2. Similarly, antibody material “specific for” VEGF-C, or that “specifically binds” VEGF-C, binds with substantially higher affinity to VEGF-C than to most or all unrelated growth factors and as compared to other vascular endothelial growth factors such as VEGF-B.

Antibody material “selective for” VEGFR-3, or that “selectively binds” VEGFR-3, binds with substantially higher affinity to VEGFR-3 than to most or all unrelated receptor tyrosine kinases such as FGFR1. Similarly, antibody material “selective for” VEGF-C, or that “selectively binds” VEGF-C, binds with substantially higher affinity to VEGF-C than to most or all unrelated growth factors. It is understood that specific and selective anti-VEGFR-3 and anti-VEGF-C antibody material can be used in the methods of the invention.

Anti-VEGFR-3 antibody material can be prepared, for example, using a VEGFR-3 fusion protein or a synthetic peptide encoding a portion of a VEGFR-3 such as SEQ ID NO: 2 as an immunogen. Similarly, anti-VEGF-C antibody material can be prepared using a VEGF-C fusion protein or a synthetic peptide encoding a portion of a VEGF-C such as SEQ ID NO: 4 as an immunogen. One skilled in the art understands that purified VEGFR-3 or VEGF-C, which can be produced recombinantly, or fragments of VEGFR-3 or VEGF-C, including peptide portions of VEGFR-3 or VEGF-C such as synthetic peptides, can be used as immunogens. Furthermore, non-immunogenic fragments or synthetic peptides of VEGFR-3 or VEGF-C can be made immunogenic by coupling the hapten to a carrier molecule such as bovine serum albumin (BSA) or keyhole limpet hemocyanin (KLH). In addition, various other carrier molecules and methods for coupling a hapten to a carrier molecule are well known in the art are described, for example, by Harlow and Lane, Antibodies: A Laboratory Manual (Cold Spring Harbor Laboratory Press, 1988)).

Anti-VEGFR-3 antibody material which binds the ligand-binding site of VEGFR-3 and inhibits ligand binding to VEGFR-3 also can be prepared by routine methods, for example, using the extracellular domain of VEGFR-3 as an immunogen, if desired, as an Fc fusion protein. Hybridomas or antibody libraries can be screened, for example, by ELISA using plates coated with 50 ng/ml of the extracellular domain of VEGFR-3 or with the same amount of the extracellular domain of another receptor such as VEGFR-2 as a control. Subsequently, positive hybridomas or library clones can be screened for VEGF-C binding inhibition, for example, with an ELISA assay using mature VEGF-C containing the N-terminal signal sequence of mouse stem cell factor and a myc epitope tag. ELISA plates coated with the extracellular domain of VEGFR-3/Fc can be incubated with various dilutions of antibodies and then with conditioned media from cells transfected with the myc-tagged VEGF-C gene. Binding with myc-tagged VEGF-C can be detected, for example, with anti-myc antibody (9E10; Santa Cruz Biotechnology; Santa Cruz, Calif.). See, for example, Kubo et al., supra, 2000.

Where substantially purified antibody material is used to prepare a pharmaceutical composition of the invention, such antibody material is substantially devoid of polypeptides, nucleic acids and other cellular material which with an antibody is normally associated in a cell. Such substantially purified antibody material also can be substantially devoid of antibody material of unrelated specificities, i.e. that does not specifically bind VEGFR-3 or that does not specifically bind VEGF-C. Antibody material can be prepared in substantially purified form, for example, by VEGFR-3 affinity purification of polyclonal anti-VEGFR-3 antisera, by screening phage displayed antibodies against a VEGFR-3 polypeptide such as SEQ ID NO: 2, or as monoclonal antibodies purified from hybridoma supernatants.

A VEGFR-3 inhibitor useful in the invention also can be a molecule that down-regulates VEGFR-3 expression, for example, a sequence-specific ribonuclease such as a ribozyme or a VEGFR-3 antisense nucleic acid molecule. Thus, the invention further provides a method of extending corneal graft survival following corneal transplantation in a patient by administering to the patient an effective amount of a pharmaceutical composition containing a VEGFR-3 inhibitor that down-regulates VEGFR-3 expression, whereby lymphangiogenesis is suppressed in the cornea of the patient.

Similarly, a VEGFR-3 inhibitor useful in the invention also can be a molecule that down-regulates VEGF-C expression, for example, a sequence-specific ribonuclease such as a ribozyme, or can be, for example, a VEGF-C antisense nucleic acid molecule. Thus, in one embodiment, the invention provides a method of extending corneal graft survival following corneal transplantation in a patient by administering to the patient an effective amount of a pharmaceutical composition containing a VEGFR-3 inhibitor that down-regulates VEGF-C expression, whereby lymphangiogenesis is suppressed in the cornea of the patient.

In further embodiments, the methods of the invention are practiced with a VEGFR-3 inhibitor which is a sequence-specific ribonuclease that down-regulates VEGFR-3 or VEGF-C expression. Such a sequence-specific ribonuclease can catalyze, for example, the specific cleavage of VEGFR-3 mRNA or VEGF-C mRNA or the mRNA of a regulatory molecule that positively modulates the expression or activity of VEGFR-3 or VEGF-C. In one embodiment, a method of the invention is practiced with a sequence-specific ribonuclease, such as a ribozyme, that down-regulates VEGFR-3 expression by cleaving VEGFR-3 RNA. In another embodiment, a method of the invention is practiced with a sequence-specific ribonuclease, such as a ribozyme, that down-regulates VEGF-C expression by cleaving VEGF-C RNA.

The term “sequence-specific ribonuclease,” as used herein, means a molecule that catalyzes the cleavage of RNA at a defined ribonucleotide sequence. A sequence-specific ribonuclease can be, for example, a ribozyme or a DNA enzyme. As used herein, the term “ribozyme” refers to a RNA molecule that catalyzes the cleavage of RNA at a defined ribonucleotide sequence.

Ribozymes such as hammerheads and hairpins can be designed and prepared by routine methods. It is understood that the specificity of ribozymes such as hammerheads and hairpins for a target cleavage site such as a site present in VEGFR-3 or VEGF-C mRNA is determined by base-pairing between the ribozyme and its RNA target. A hammerhead ribozyme, for example, cleaves after “UX” dinucleotides, where X is any ribonucleotide except guanosine, with a higher rate of cleavage when X is cytosine. “NUX” triplets generally are present in the target sequence, where N is any ribonucleotide, and GUC, CUC or UUC triplets are often present in the target RNA. Two stretches of antisense sequence 6-8 nucleotides long that flank the 21 nucleotide sequence forming the catalytic hammerhead between them are then designed based on the target sequence surrounding the third nucleotide (“X”) of the triplet. This nucleotide is not based paired with the ribozyme. Methods of designing hammerhead ribozymes are well known as described, for example, in Hauswirth and Lewin, Prog. Retin. Eye Res. 19:689-710 (2000), and Lewin and Hauswirth, Trends. Mol. Med. 7:221-228 (2001).

Hairpin ribozymes also are well known in the art and can be useful in extending corneal graft survival according to a method of the invention. Hairpin ribozymes have a catalytic core of about 34 nucleotides and recognize the sequence NNYNGUCNNNNNN (SEQ ID NO: 6), where N is any nucleotide and Y is a pyrimidine. The “NGUC” (SEQ ID NO: 7) sequence is not base-paired with the ribozyme. In one embodiment, a method of the invention is practiced with a hairpin ribozyme that recognizes a “NGUC” (SEQ ID NO: 7) motif present, for example, in a VEGFR-3 or VEGF-C mRNA. In further embodiments, a method of the invention relies on a hairpin ribozyme having a tetraloop in the catalytic core rather than a 3-base loop, or a U to C substitution at position 39 of the catalytic core, or both (Hauswirth and Lewin, supra, 2000; and Lewin and Hauswirth, supra, 2001).

One skilled in the art understands that target sequences, for example, in VEGFR-3 or VEGF-C mRNA generally are selected to avoid secondary structures, which can interfere with the ability of a ribozyme to bind to the target site. Well-known structure-predicting algorithms can be used; in addition, potential ribozymes can be evaluated, if desired, for accessibility to hybridization with complementary sequences using a ribonuclease protection assay. The nucleotide sequences encoding human VEGFR-3 and human VEGF-C are disclosed herein as SEQ ID NO: 1 and SEQ ID NO: 3, respectively. Additional nucleotide sequences encoding species homologs also are well known in the art, as described, for example, in Finnerty et al., supra, 1993; and Eichmann et al., supra, 1996.

Sequence-specific ribonucleases, including ribozymes and DNA enzymes, can be designed as described above and prepared by standard methods for synthesis of nucleic acid molecules. See, also, Ke et al., Int. J. Oncol. 12:1391-1396 (1998); Doherty et al., Ann. Rev. Biophys. Biomol. Struct. 30:457-475 (2001); Hauswirth and Lewin, supra, 2000; and Lewin and Hauswirth, supra, 2001. Sequence-specific ribozymes also can be identified by in vitro selection from pools of random sequences. Such methods are well-established, as described, for example, in Bartel and Szostak, Science 261:1411-1418 (1993), Breaker, Chem. Rev. 97:371-390 (1997) and Santoro and Joyce, Proc. Natl. Acad. Sci., USA 94:4262-4266 (1997)).

Where a ribozyme is to be administered to a patient without being delivered using a viral or other vector, the ribozyme can be modified, if desired, to enhance stability. Modifications useful in a therapeutic ribozyme include, but are not limited to, blocking the 3′ end of the molecule and the 2′ positions of pyrimidines. Stabilized ribozymes can have half-lives of hours and can be administered repeatedly using, for example, intravenous or topical injection. Those skilled in the art understand that a ribozyme also can be administered by expression in a viral gene therapy vector. A DNA oligonucleotide encoding the ribozyme can be cloned downstream of a RNA pol II or RNA pol III promoter and, if desired, can be embedded within the transcripts of genes such as tRNAᵥₐₗ, U6 snRNA or the adenoviral VA1 RNA.

A VEGFR-3 inhibitor useful in the methods of the invention also can be an antisense nucleic acid molecule that down-regulates VEGFR-3 or VEGF-C expression. Such an antisense nucleic acid molecule can reduce mRNA translation or increase mRNA degradation of VEGFR-3 or VEGF-C mRNA or the mRNA of a regulatory molecule that positively modulates the expression or activity of VEGFR-3 or VEGF-C. In one embodiment, a method of the invention is practiced with a pharmaceutical composition containing a VEGFR-3 antisense nucleic acid molecule. In another embodiment, a method of the invention is practiced with a pharmaceutical composition containing a VEGF-C antisense nucleic acid molecule.

The term “antisense nucleic acid molecule” as used herein, means a nucleic acid molecule that is complementary in sequence to all or part of a molecule of messenger RNA or another specific RNA transcript. Thus, a VEGFR-3 antisense nucleic acid molecule is complementary to some or all of a VEGFR-3 mRNA such as a human VEGFR-3 mRNA. Similarly, a VEGF-C antisense nucleic acid molecule is complementary to some or all of a VEGF-C mRNA such as a human VEGF-C mRNA. An antisense nucleic acid molecule can be, for example, DNA or RNA, and can include naturally occurring nucleotides as well as synthetic nucleotides or other non-naturally occurring modifications such as modifications to the phosphate backbone that improve stability. Antisense oligonucleotides, including phosphorothioate and other modified oligonucleotides, are encompassed by the term antisense nucleic acid molecule as used herein.

Without being bound by the following, an antisense nucleic acid molecule useful in the invention can reduce mRNA translation or increase mRNA degradation, thereby reducing expression of the target mRNA such as human VEGFR-3 or VEGF-C mRNA. It is understood that an antisense nucleic acid molecule can be perfectly complementary to a target nucleic acid sequence, for example, in a VEGFR-3 or VEGF-C mRNA such as human VEGFR-3 mRNA or human VEGF-C mRNA, or can contain one or mismatches relative to the patient's endogenous nucleic acid sequence. The homology requirement for reduction of expression using antisense methodology can be determined empirically. Generally, at least about 80-90% nucleic acid sequence identity is present in an antisense nucleic acid molecule useful in the invention, with higher nucleic acid sequence identity often used in antisense oligonucleotides, which can be perfectly identical to the patient's endogenous transcript. The target sequence can be chosen, if desired, to have a small single-stranded region at which nucleation takes place, in addition to a double-stranded, helically ordered stem that is invaded by the antisense molecule to displace one of the strands (Mir and Southern, Nature Biotech. 17:788-792 (1999). Methods for selecting and preparing antisense nucleic acid molecules are well known in the art and include in silico approaches (Patzel et al. Nucl. Acids Res. 27:4328-4334 (1999); Cheng et al., Proc. Natl. Acad. Sci., USA 93:8502-8507 (1996); Lebedeva and Stein, Ann. Rev. Pharmacol. Toxicol. 41:403-419 (2001); Juliano and Yoo, Curr. Opin. Mol. Ther. 2:297-303 (2000); and Cho-Chung, Pharmacol. Ther. 82:437-449 (1999)).

An antisense nucleic acid molecule can include, for example, at least 10 contiguous nucleotides complementary to the human VEGFR-3 sequence shown as SEQ ID NO: 1, or another VEGFR-3 encoding sequence or control sequence or a 5′ or 3′ untranslated sequence. An antisense nucleic acid molecule also can include, for example, at least 15, 20, 25, 30, 35, 40, 45, 50, 100, 200, 300, 500 or more contiguous nucleotides complementary to SEQ ID NO: 1 or another VEGFR-3 encoding sequence or control sequence or a 5′ or 3′ untranslated sequence. If desired, an antisense nucleic acid molecule can be complementary to the full-length of the target message. Similarly, an antisense nucleic acid molecule useful in the invention can include, for example, at least 10, 15, 20, 25, 30, 35, 40, 45, 50, 100, 200, 300 or more contiguous nucleotides complementary to the human VEGF-C sequence shown as SEQ ID NO: 3 or another VEGF-C encoding sequence or control sequence or a 5′ or 3′ untranslated sequence. Antisense oligonucleotides useful in the invention, including phosphorothioate and other oligonucleotides with otherwise modified backbones, can have, for example, from 12 to 100 nucleotides, for example, from 12 to 50 or from 12 to 30 nucleotides, or from 15 to 100, 15 to 50, or 15 to 30 nucleotides, or from 20 to 100, 20 to 50, or 20 to 30 nucleotides complementary to VEGFR-3 or VEGF-C, for example, complementary to the human VEGFR-3 sequence shown as SEQ ID NO: 1 or the human VEGF-C sequence shown as SEQ ID NO: 3. Antisense oligonucleotides useful in the invention can have, for example, 12, 13, 14, 15, 16, 17, 18, 19, 20, 21, 22, 23, 24, 25, 26, 27, 28, 29 or 30 nucleotides complementary, for example, to the human VEGFR-3 sequence shown as SEQ ID NO: 1 or the human VEGF-C sequence shown as SEQ ID NO: 3.

In one embodiment, the antisense nucleic acid molecule is a nuclease-resistant nucleic acid molecule with a modified backbone such as a phosphorothiorate oligodeoxynucleotide, in which a sulfur atom is substituted for a nonbridging oxygen at each phosphorus. Antisense nucleic acid molecules useful in the invention further include mixed backbone oligonucleotides such as phosphorothioate oligodeoxynucleotides containing segments of 2′-O-methyloligoribonucleotides (2′-O-meRNA) or methylphosphonate oligodeoxynucleotides (me-PDNA), which are more resistant to nucleases and form more stable duplexes with RNA than the corresponding phosphorothioate oligodeoxynucleotide (Cho-Chung, supra, 1999). Antisense nucleic acid molecules useful in the invention also include chimeric antisense oligonucleotides (denoted “gap-mers”) containing a “central core” of several consecutive oligodeoxy-containing bases and 2′-O-alkylloligoribonucleotide (methyl or methoxyethoxy) modifications incorporated into the remaining bases, with the backbone composed entirely of phosphorothioate linkages. For example, a central core of 6 to 8 oligodeoxyribonucleotides can be flanked by 6 to 8 2′-O-alkylloligoribonucleotides at the 5′ and 3′ ends.

While not wishing to be bound by the following, antisense activity can result from cleavage of the mRNA strand by RNase H at the site of hybridization. Thus, in one embodiment, the antisense nucleic acid molecule includes a backbone portion that is RNase H competent. Such competent backbones have phosphodiester or phosphorothioate linkages and deoxyribose sugar moieties. Uncharged backbones, for example, methylphosphonate or peptide nucleic acid linkages, or 2′-O-methylribose or another substitution at the 2′ position, are not competent for cleavage by RNase H.

A VEGFR-3 inhibitor useful in the invention also can be an inhibitor of the intracellular signaling that occurs upon VEGFR-3 stimulation. VEGFR-3 signaling begins with VEGF-C or VEGF-D binding to the second immunoglobulin-homology domain of VEGFR-3, with subsequent receptor dimerization and transphosphorylation. The long VEGFR-3 isoform is autophosphorylated to a greater extent than the short isoform, and the two isoforms also differ in their signaling properties, with the long isoform able to mediate cell growth in soft agar and tumorigenicity in nude mice (Fournier et al., Oncogene 11:921-931 (1995); Pajusola et al., supra, 1993; Karkkainen and Petrova, Oncogene 19:5598-5605 (2000); and Petrova et al., Exper. Cell Res. 253:117-130 (1999)).

Stimulation with VEGFR-3 ligand also induces rapid tyrosine phosphorylation of the Shc protein. Shc phosphorylation levels are higher in cells expressing the long isoform of VEGFR-3, and mutation of Tyr1377, which is only present in the long isoform, to phenylalanine reduces Shc phosphorylation and prevents tumorigenic cell transformation by VEGFR-3. Shc appears to serve as a negative regulator of VEGFR-3 activity, because mutations of Shc phosphorylation sites lead to increased transforming activity of VEGFR-3 (Fournier et al., 18:507-514 (1999)). In addition, both VEGFR-3 isoforms bind in a ligand-dependent manner to the SH2 domains of Grb2 and PLCγ but not to the SH2 domain of PI3-K (Fournier et al., supra, 1995; Pajusola et al., Oncogene 9:3545-3555 (1994); and Founier et al., J. Biol. Chem. 271:12956-12963 (1996)).

Results obtained in a human erythroleukemia cell line that expresses high levels of VEGFR-3 indicate that VEGF-C stimulation induces cell growth and recruitment of the signaling molecules Shc, Grb2 and human son of sevenless (hSOS) to activated VEGFR-3 (Wang et al., Blood 90:3507-3515 (1997)). In addition, VEGF-C stimulation induces tyrosine phosphorylation of paxillin, a cytoskeletal protein, and results in an increased association of paxillin with related adhesion focal tyrosine kinase (RAFTK). c-Jun NH₂-terminal kinase (JNK) also can be activated following VEGF-C stimulation (Liu et al., J. Clin. Invest. 99:1798-1804 (1997)). Furthermore, tyrosine phosphorylation of Shc leads to activation of the mitogen activated protein kinases, ERK1 and ERK2 (see FIG. 1).

Thus, a VEGFR-3 inhibitor can be an inhibitor of VEGFR-3 intracellular signaling that acts by modulating, for example, recruitment, expression or activity of Shc, Grb2, hSOS or PLCγ. A VEGFR-3 inhibitor also can effect VEGFR-3 intracellular signaling, for example, by modulating the association of paxillin with RAFTK or by modulating the expression or activity of paxillin or RAFTK. Similarly, an inhibitor of VEGFR-3 intracellular signaling can modulate the recruitment, expression or activity of JNK, or the recruitment, expression or activity of ERK1 or ERK2. As used herein, the term “inhibitor of VEGFR-3 intracellular signaling” means a molecule that acts to reduce one or more cellular responses to VEGF-C binding to VEGFR-3 down stream of VEGFR-3 and without directly effecting the expression or activity of VEGFR-3. It is understood that an inhibitor of VEGFR-3 intracellular signaling can act positively or negatively on a component of the VEGFR-3 intracellular pathway and that such an inhibitor can be, without limitation, a small molecule, ATP analog, protein or nucleic acid molecule, including a dominant negative protein, kinase inhibitor, ribozyme or antisense molecule. As an example, an inhibitor of VEGFR-3 intracellular signaling can be a molecule that enhances the recruitment, expression or activity of Shc, since Shc is a negative regulator of VEGFR-3 signaling.

An inhibitor of VEGFR-3 intracellular signaling can be a specific, selective or non-selective inhibitor. Such a selective inhibitor reduces VEGFR-3 signaling in preference to the signaling induced by most or all unrelated receptor tyrosine kinases such as FGFR1. A specific inhibitor of VEGFR-3 intracellular signaling reduces VEGFR-3 signaling in preference to the signaling of most or all unrelated receptor tyrosine kinases such as FGFR1 and in preference to the vascular endothelial growth factor receptors VEGFR-1 and VEGFR-2. A non-selective inhibitor of VEGFR-3 intracellular signaling reduces the signaling of other tyrosine kinase receptors and one or all other vascular endothelial growth factor receptors to a similar extent as the signaling induced by VEGFR-3. One skilled in the art understands that specific, selective and non-selective inhibitors of VEGFR-3 intracellular signaling can be useful for extending corneal graft survival, according to the methods disclosed herein.

The invention also provides methods of extending corneal graft survival following corneal transplantation in a patient by administering to the patient an anti-lymphangiogenic agent, whereby lymphangiogenesis is suppressed in the cornea of the patient. As used herein, the term “anti-lymphangiogenic agent” means a molecule that reduces or inhibits the sprouting or formation of new lymphatic vessels from pre-existing vessels. Such an anti-lymphangiogenic agent can be, for example, a VEGFR-3 inhibitor or an inhibitor of another molecule that functions in nature to promote lymphangiogenesis. As described above in regard to VEGFR-3 inhibitors, such a molecule can be, without limitation, a dominant negative inhibitor, a sequence-specific ribonuclease, an antisense molecule, an antibody, a small molecule inhibitor or an inhibitor of an intracellular pathway that is normally activated by the lymphangiogenic agent.

In one embodiment, corneal graft survival also is extended by administering to the patient an anti-angiogenic agent in addition to the pharmaceutical composition containing the VEGFR-3 inhibitor. In another embodiment, an immunosuppressive agent is administered to the patient in addition to the pharmaceutical composition containing the VEGFR-3 inhibitor and, if desired, in conjunction with administration of an anti-angiogenic agent.

The term “anti-angiogenic agent,” as used herein, means a molecule that reduces or inhibits angiogenesis. It is understood that the anti-angiogenic agent and VEGFR-3 inhibitor, or other anti-lymphangiogenic agent, can be administered independently or simultaneously, in the same or different pharmaceutical compositions, and by the same or different routes of administration. In one embodiment, the invention is practiced by administering a bi-functional molecule having both anti-lymphangiogenic and anti-angiogenic activity. In a further embodiment, the invention is practiced by administering a bi-functional molecule that contains a VEGFR-3 inhibitor and anti-angiogenic agent.

A variety of anti-angiogenic agents useful in the invention are known in the art and can be prepared by routine methods. See, for example, Hagedorn and Bikfalvi, Crit. Rev. Oncol. Hematol. 34:89-110 (2000) and Kirsch et al., J. Neurooncol. 50:149-163 (2000). Anti-angiogenic agents include, without limitation, small molecules; proteins such as angiogenic factors and receptors, transcription factors, and antibodies and antigen-binding fragments thereof; peptides and peptidomimetics; and nucleic acid molecules including ribozymes, antisense oligonucleotides, and nucleic acid molecules encoding, for example, dominant negative angiogenic factors and receptors, transcription factors, and antibodies and antigen-binding fragments thereof.

An anti-angiogenic agent can be, for example, an inhibitor or neutralizing antibody that reduces the expression or signaling of an angiogenic factor such as vascular endothelial growth factor (VEGF), which is a major inducer of angiogenesis in normal and pathological conditions, and is essential in embryonic vasculogenesis. The biological effects of VEGF include stimulation of endothelial cell proliferation, survival, migration and tube formation, and regulation of vascular permeability. An anti-angiogenic agent also can inhibit another angiogenic factor such as a member of the fibroblast growth factor (FGF) family such as FGF-1 (acidic), FGF-2 (basic), FGF-4 or FGF-5 (Slavin et al., Cell Biol. Int. 19:431-444 (1995); Folkman and Shing, J. Biol. Chem. 267:10931-10934 (1992)) or angiopoietin-1, a factor that signals through the endothelial cell-specific Tie2 receptor tyrosine kinase (Davis et al., Cell 87:1161-1169 (1996); and Suri et al., Cell 87:1171-1180 (1996)), or the receptor of one of these angiogenic factors. It is understood that a variety of mechanisms can act to inhibit activity of an angiogenic factor including, without limitation, direct inhibition of receptor binding, indirect inhibition by reducing secretion of the angiogenic factor into the extracellular space, or inhibition of signaling, expression or function of the angiogenic factor.

A variety of other molecules also can function as anti-angiogenic agents useful in the invention including, without limitation, angiostatin; endostatin; heparin-binding fragments of fibronectin; a modified form of antithrombin; collagenase inhibitors; basement membrane turnover inhibitors; angiostatic steroids; platelet factor 4, and fragments and peptides thereof; thrombospondin, and fragments and peptides thereof; and doxorubicin (O'Reilly et al., Cell 79:315-328 (1994)); O'Reilly et al., Cell 88: 277-285 (1997); Homandberg et al., Am. J. Path. 120:327-332 (1985); Biochim. Biophys. Acta 874:61-71 (1986); and O'Reilly et al., Science 285:1926-1928 (1999)).

Exemplary anti-angiogenic agents useful in the invention include, yet are not limited to, angiostatin, endostatin, metastatin and 2ME2 (EntreMed; Rockville, Md.); anti-VEGF antibodies such as Avastin (Genentech; South San Francisco, Calif.); and VEGFR-2 inhibitors such as SU5416, a small molecule inhibitor of VEGFR-2 (SUGEN; South San Francisco, Calif.) and SU6668 (SUGEN), a small molecule inhibitor of VEGFR-2, platelet derived growth factor and fibroblast growth factor I receptor. It is understood that these as well as other anti-angiogenic agents well known in the art or that can be prepared by routine methods are encompassed by the term “anti-angiogenic agent” and can be used to extend corneal graft survival according to a method of the invention.

An immunosuppressive agent also can be administered to the corneal transplantation patient in addition to the VEGFR-3 inhibitor or other anti-lymphangiogenic agent. Such immunosuppressive agents can be useful, for example, for treating a corneal transplantation patient with an elevated risk of allograft rejection or a patient exhibiting one or more symptoms consistent with allograft rejection. Immunosuppressive agents useful in the methods of the invention encompass, without limitation, steroids such corticosteroids; the steroid prednisolone acetate; cyclosporin and tacrolimus (FK506); and therapeutic monoclonal antibodies such as anti-T lymphocyte, anti-CD4+ cell, anti-ICAM-1 and anti-IL-2 antibodies.

A corticosteroid immunosuppressive agent can be administered, for example, topically, periocularly, systemically, or using multiple routes of administration. For example, prednisolone acetate can be administered topically as a 1% preparation. Topical prednisolone acetate can be applied hourly for mild reactions combined with intravenous methylprednisolone pulse therapy (3 to 5 mg/kg IV push) followed by 5 days of oral prednisone (1 mg/kg/day) for severe reactions. A single dose of intravenous methylprednisolone (500 mg) can be substituted, if desired, for daily oral prednisone (60 to 80 mg) when combined with topical therapy. One skilled in the art understands that these and other corticosteroid immunosuppressive agents can be useful in the methods of the invention.

The immunosuppressive agent cyclosporin also can be useful in the methods of the invention and can be administered systemically for a period of, for example, months or years, or can be administered topically, for example, as a 2% cyclosporin formulation. Therapeutic monoclonal antibodies also can be useful in the methods of the invention; for example, anti-T lymphocyte or other immunosuppressive monoclonal antibodies can be administered intracamerally. It is understood that these and other immunosuppressive agents can be administered, as desired, in combination with a pharmaceutical composition containing an anti-VEGFR-3 inhibitor according to a method of the invention.

In the methods of the invention, a pharmaceutical composition containing a VEGFR-3 inhibitor can be administered prior to, during, or subsequent to corneal transplantation. If desired, administration of the pharmaceutical composition containing the VEGFR-3 inhibitor can be administered repeatedly as needed. In one embodiment, administration is repeated over a period of at least one month. In another embodiment, administration is repeated over a period of at least six months.

In a further embodiment, the invention provides a method of extending corneal graft survival following corneal transplantation in a patient by administering to the patient prior to corneal transplantation an effective amount of a pharmaceutical composition containing a VEGFR-3 inhibitor; and administering to the patient subsequent to corneal transplantation an effective amount of a pharmaceutical composition containing a VEGFR-3 inhibitor, whereby lymphangiogenesis is suppressed in the cornea of the patient. The pre- and post-surgical pharmaceutical compositions can be the same or different and can be administered using the same or different routes of delivery.

It is understood that a pharmaceutical composition containing a VEGFR-3 inhibitor or other anti-lymphangiogenic agent can be administered prior to corneal transplantation, during corneal transplantation, or subsequent to corneal transplantation, or at a combination of these times. It further is understood that a pharmaceutical composition containing a VEGFR-3 inhibitor or other anti-lymphangiogenic agent can be administered prior to the onset of symptoms of allograft rejection, for example, as a routine precaution for all patients prior to, during or subsequent to surgery, or can be administered selectively in high risk patients, for example, those with a history of graft rejection. Administration can be repeated, for example, over a period of two weeks, one month, two months, three months, four months, five months, six months, one year or two years, as often as necessary to maintain the beneficial effect of the anti-lymphangiogenic agent. Those skilled in the art recognize that the frequency of administration depends on the precise nature of the VEGFR-3 inhibitor or other anti-lymphangiogenic agent, as well as the concentration at which it is administered, and the extended release formulation used, if any. An ophthalmic composition useful in a method of the invention can be administered, for example, once or twice daily, or three or four times daily. It is understood that during critical periods, such as immediately post-surgery or upon the occurrence of one or more symptoms of allograft rejection, an ophthalmic composition such as a topical ophthalmic composition can be administered more frequently, for example, on an hourly basis.

In a method of the invention, the VEGFR-3 inhibitor or other anti-lymphangiogenic agent is administered in a pharmaceutical composition. A pharmaceutical composition useful in the invention includes a VEGFR-3 inhibitor or other anti-lymphangiogenic agent in a concentration range of, for example, approximately 0.0001% to approximately 0.1% weight by volume. A pharmaceutical composition useful in the methods of the invention further can include an excipient well known in the art for preparing pharmaceutical compositions such as ophthalmic compositions.

In accordance with the invention, the VEGFR-3 inhibitor or other anti-lymphangiogenic agent is administered in sufficient concentration so as to deliver an effective amount of the inhibitor or agent to the eye. An ophthalmic solution generally contains, for example, VEGFR-3 inhibitor or other anti-lymphangiogenic agent in a concentration range of approximately 0.0001% to approximately 0.1% (weight by volume), for example, approximately 0.0005% to approximately 0.1% (weight by volume).

The VEGFR-3 inhibitor or other anti-lymphangiogenic agent can be administered, if desired, in an ophthalmic composition containing an ophthalmically acceptable carrier, which is any carrier that has substantially no long term or permanent detrimental effect on the eye to which it is administered. Examples of ophthalmically acceptable carriers include, without limitation, water, such as distilled or deionized water; saline; and other aqueous media. In one embodiment, the ophthalmic composition is an ophthalmic solution containing a soluble anti-lymphangiogenic agent such as a soluble VEGFR-3 inhibitor. In another embodiment, the ophthalmic composition contains the VEGFR-3 inhibitor or other anti-lymphangiogenic agent as a suspension in a suitable carrier.

Topical ophthalmic compositions can be useful in the methods of the invention for extending corneal graft survival and include, without limitation, ocular drops, ocular ointments, ocular gels and ocular creams. Such ophthalmic compositions are easy to apply and deliver the active ingredient effectively and avoid possible systemic side effects.

The components of an exemplary topical composition are shown below in Table 1.

TABLE I

| Ingredient | Amount (% W/V) |
|---|---|
| VEGFR-3 inhibitor or | about 0.0001 to |
| anti-lymphangiogenic agent | about 0.1 |
| preservative | 0-0.10 |
| Vehicle | 0-40 |
| Tonicity Adjustor | 1-10 |
| Buffer | 0.01-10 |
| pH Adjustor | q.s. pH 4.5-7.5 |
| antioxidant | As needed |
| Purified Water | As needed to make 100% |

A preservative can be included, if desired, in an ophthalmic composition useful in the invention, such as the topical composition shown in Table 1. Such preservatives include, without limitation, benzalkonium chloride, chlorobutanol, thimerosal, phenylmercuric acetate, and phenylmercuric nitrate. Vehicles useful in a topical ophthalmic composition include, yet are not limited to, polyvinyl alcohol, povidone, hydroxypropyl methyl cellulose, poloxamers, carboxymethyl cellulose, hydroxyethyl cellulose and purified water.

A tonicity adjustor can be included, if desired, in an ophthalmic composition administered to extend corneal graft survival according to a method of the invention. Such a tonicity adjustor can be, for example, a salt such as sodium chloride, potassium chloride, mannitol or glycerin, or another pharmaceutically or ophthalmically acceptable tonicity adjustor.

Various buffers and means for adjusting pH can be used to prepare an ophthalmic composition useful in the invention, provided that the resulting preparation is ophthalmically acceptable. Such buffers include, without limitation, acetate buffers, citrate buffers, phosphate buffers and borate buffers. It is understood that acids or bases can be used to adjust the pH of the composition as needed. Ophthalmically acceptable antioxidants useful in preparing an ophthalmic composition include, yet are not limited to, sodium metabisulfite, sodium thiosulfate, acetylcysteine, butylated hydroxyanisole and butylated hydroxytoluene.

A VEGFR-3 inhibitor or other anti-lymphangiogenic agent can be administered to a patient by a variety of means depending, in part, on the type of agent to be administered and the history, risk factors and symptoms of the patient. Routes of administration suitable for the methods of the invention include both systemic and local administration. Thus, in one embodiment, a method of the invention for extending corneal graft survival is practiced by systemic administration of a pharmaceutical composition containing a VEGFR-3 inhibitor or other anti-lymphangiogenic agent. In another embodiment, a method of the invention is practiced by local administration of a pharmaceutical composition containing an anti-lymphangiogenic agent such as a VEGFR-3 inhibitor. In further embodiments, a pharmaceutical composition containing the VEGFR-3 inhibitor or other anti-lymphangiogenic agent is administered topically, or by local injection, or is released from an intraocular or periocular implant.

As used herein, the term “systemic administration” means a mode of administration resulting in delivery of a pharmaceutical composition to essentially the whole body of the patient. Exemplary modes of systemic administration include, without limitation, intravenous injection and oral administration. The term “local administration,” as used herein, means a mode of administration resulting in significantly more pharmaceutical composition being delivered to and about the eyes than to regions distal from the eyes.

Systemic and local routes of administration useful in the methods of the invention encompass, without limitation, oral gavage; intravenous injection; intraperitoneal injection; intramuscular injection; subcutaneous injection; transdermal diffusion and electrophoresis; topical eye drops and ointments; periocular and intraocular injection including subconjunctival injection; extended release delivery devices including locally implanted extended release devices; and intraocular and periocular implants including bioerodible and reservoir-based implants.

In one embodiment, an ophthalmic composition containing a VEGFR-3 inhibitor or other anti-lymphangiogenic agent is administered topically to the eye. The ophthalmic composition can be for example, an ophthalmic solution (ocular drops). In another embodiment, an ophthalmic composition containing VEGFR-3 inhibitor or other anti-lymphangiogenic agent is injected directly into the eye. In a further embodiment, an ophthalmic composition containing the VEGFR-3 inhibitor or other anti-lymphangiogenic agent is released from an intraocular or periocular implant such as a bioerodible or reservoir-based implant.

In one embodiment, an ophthalmic composition containing a VEGFR-3 inhibitor or other anti-lymphangiogenic agent is administered locally in an extended release formulation. For example, an ophthalmic composition containing a VEGFR-3 inhibitor or other anti-lymphangiogenic agent can be administered via an intraocular or periocular implant, which can be, for example, bioerodible or reservoir-based. As used herein, the term “implant” refers to any material that does not significantly migrate from the insertion site following implantation. An implant can be biodegradable, non-biodegradable, or composed of both biodegradable and non-biodegradable materials; a non-biodegradable implant can include, if desired, a refillable reservoir. Implants useful in the methods of the invention include, for example, patches, particles, sheets, plaques, microcapsules and the like, and can be of any shape and size compatible with the selected site of insertion, which can be, without limitation, the posterior chamber, anterior chamber, suprachoroid or subconjunctiva. It is understood that an implant useful in the invention generally releases the implanted pharmaceutical composition at an effective dosage to the cornea of the patient over an extended period of time. A variety of ocular implants and extended release formulations suitable for ocular release are well known in the art, as described, for example, in U.S. Pat. Nos. 5,869,079 and 5,443,505.

Where a VEGFR-3 inhibitor or other anti-lymphangiogenic is a nucleic acid molecule, administration of a pharmaceutical composition containing the nucleic acid molecule can be carried out using one of numerous methods well known in the art of gene therapy. Such methods include, but are not limited to, ballistic gun delivery, lentiviral transformation, adenoviral transformation, cytomegaloviral transformation, microinjection and electroporation as described further below.

As an example, ballistic gun delivery can be useful in the methods of the invention for extending corneal graft survival and can be performed as described in Tanelian et al., BioTechniques, 23:484-488 (1997), to achieve focal delivery and expression of a plasmid in corneal epithelium with high efficiency. In this method, 0.2-0.5 mg gold particles are coated with plasmid DNA, which is then delivered into cornea using a ballistic gun. The depth of delivery of the plasmid DNA is a function of the pressure of the gun, thus facilitating delivery of plasmid DNA to a desired depth.

A lentivirus also can be used to administer a pharmaceutical composition containing a nucleic acid molecule according to a method of the invention. Cells can be transduced with lentivirus in vitro or in situ as described, for example, in Wang et al., Gene Therapy 7:196-200 (2000). Corneal endothelial cells, epithelial cells and stromal keratocytes in human cornea can be exposed to a lentivirus that includes a nucleic acid molecule which is an anti-lymphangiogenic agent such as a VEGFR-3 inhibitor. Exposed cells can continue to express the encoded protein for at least 60 days after transduction.

An adenovirus also can be used to administer a nucleic acid molecule to the cornea in vivo after surgical removal of superficial epithelial cells from the cornea. For example, adenovirus can be administered to the anterior chamber of the eye. Procedures for administration of adenovirus are well known in the art, as described, for example, in U.S. Pat. No. 5,827,702.

Microinjection and electric pulse also can be used to administer a pharmaceutical composition which contains a nucleic acid molecule that is a VEGFR-3 inhibitor or other anti-lymphangiogenic agent. Microinjection and electric pulse can be used, for example, to introduce cytomegalovirus, or a plasmid expression vector, into cornea (Sakamoto et al., Hum. Gene Ther. 10:2551-2557 (1999), and Oshima et al., Gene Therapy 5:1347-1354 (1998)). Injection of virus or plasmid into the anterior chamber at the limbus, followed by electric pulses, results in transduction of corneal endothelial cells. It is understood that these and other methods can be used, as desired, to administer a pharmaceutical composition in which the VEGFR-3 inhibitor or other anti-lymphangiogenic agent is a nucleic acid molecule.

The following examples are intended to illustrate but not limit the present invention.

### EXAMPLE I

### Increased Corneal Graft Survival in Animals Treated with Inhibitors of Lymphangiogenesis

Grafts are prepared and transferred according to the well-characterized rat model of keratoplasty with transplantation of corneas from Lewis strain rats to Wistar-Furth recipients (Callanan et al., Transplantation 45:437-443 (1988)). Each treatment group administered vehicle or test agent includes nine to fourteen rats. Grafts are observed clinically and scored three times per week for signs of rejection according to the criteria in Callanan et al., supra, 1988. Day 60 following surgery represents a two-fold prolongation in the expected mean survival time for corneal transplants in the Lewis/Wistar-Furth combination and therefore is selected as an advantageous time for terminating treatment. Rats bearing grafts not rejected by day 60 are observed for an additional 14 days to determine if the host's immune system has been tolerized. At this time, 80% of the grafted eyes are snap frozen for cryostat sectioning, and the remaining 20% of the eyes are fixed in formalin for H & E staining.

3(2,4-dihydroxy-benzylidene)-1,3-dihydro-indol-2-one (MAE87), 3-(3-fluoro-4-methoxy-benzylidene)-1,3-dihydro-indol-2-one (MAE106) and 3-(4-dimethylamino-naphthalen-1-ylmethylene)-1,3-dihydro-indol-2-one (MAZ51) were prepared essentially as follows. Indolin-2-one (10 mmol) is mixed with 10 mmol of either 2,4-dihydroxy-benzaldehyde (MAE87), 3-fluoro-4-methoxy-benzaldehyde (MAE 106) or 4-dimethylamino-naphthalene-1-carbaldehyde (MAZ51). The reactions are refluxed for 5 hours with three drops piperidine in 40 mL ethanol (Kirkin et al., supra, 2001). The products are filtered, washed with ethanol and dried under vacuum. The structures are shown below in Table 2. The melting point of MAE87 is 250° C.; the melting point of MAE106 is 220° C.; and the melting point of MAZ51 is greater than 250° C.

TABLE 2
MAE87

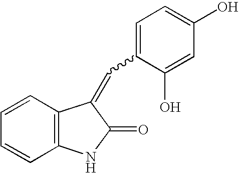

MAE106

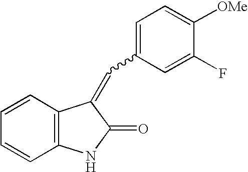

MAZ51

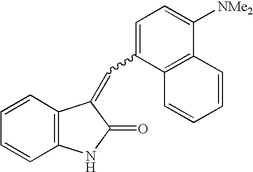

The VEGFR-3 tyrosine kinase inhibitor MAE87, MAE106 or MAZ51 is administered systemically at various concentrations, ranging from 0.5 to 200 mg/kg/day. In other animals, the compound is administered as an eye drop solution in various concentrations ranging from 0.05% to 5.0% and administered as various frequencies (once per day, two times per day and three times per day).

Animals receiving only vehicle demonstrate evidence of graft rejection, on average, at day 30. In contrast, in animals receiving MAE87, MAE106 or MAZ51 exhibit increased mean graft survival as demonstrated by a significant delay in evidence of graft rejection.

These results demonstrate that inhibitors of VEGFR-3 tyrosine kinase activity act to increase mean corneal graft survival time in a well-accepted rat model of keratoplasty.

All journal article, reference and patent citations provided above, in parentheses or otherwise, whether previously stated or not, are incorporated herein by reference in their entirety.

Although the invention has been described with reference to the examples provided above, it should be understood that various modifications can be made without departing from the spirit of the invention. Accordingly, the invention is limited only by the claims.

We claim:

1. A method of extending corneal graft survival following corneal transplantation in a patient, comprising:
administering to said patient an effective amount of a pharmaceutical composition comprising a vascular endothelial growth factor receptor-3 (VEGFR-3) inhibitor,
whereby lymphangiogenesis is suppressed in the cornea of said patient.

2. The method of claim 1, wherein said VEGFR-3 inhibitor is a dominant negative VEGFR-3 receptor.

3. The method of claim 2, wherein said dominant negative VEGFR-3 receptor is kinase-inactive.

4. The method of claim 2, wherein said dominant negative VEGFR-3 receptor is soluble.

5. The method of claim 1, wherein said VEGFR-3 inhibitor is a nucleic acid molecule encoding a dominant negative VEGFR-3 receptor.

6. The method of claim 5, wherein said dominant negative VEGFR-3 receptor is kinase-inactive.

7. The method of claim 5, wherein said dominant negative VEGFR-3 receptor is soluble.

8. The method of claim 1, wherein said VEGFR-3 inhibitor is a VEGFR-3 kinase inhibitor.

9. The method of claim 8, wherein said VEGFR-3 kinase inhibitor binds the VEGFR-3 catalytic domain.

10. The method of claim 9, wherein said VEGFR-3 kinase inhibitor is an ATP analog.

11. The method of claim 1, wherein said VEGFR-3 inhibitor is a VEGFR-3 binding molecule.

12. The method of claim 11, wherein said VEGFR-3 binding molecule binds the VEGFR-3 extracellular domain.

13. The method of claim 11, wherein said VEGFR-3 binding molecule is anti-VEGFR-3 antibody material.

14. The method of claim 13, wherein said anti-VEGFR-3 antibody material is monoclonal.

15. The method of claim 1, wherein said VEGFR-3 inhibitor down-regulates VEGFR-3 expression.

16. The method of claim 15, wherein said VEGFR-3 inhibitor is a sequence-specific ribonuclease.

17. The method of claim 16, wherein said sequence-specific ribonuclease is a ribozyme.

18. The method of claim 15, wherein said VEGFR-3 inhibitor is a VEGFR-3 antisense nucleic acid molecule.

19. The method of claim 1, wherein said VEGFR-3 inhibitor is anti-VEGF-C neutralizing antibody material.

20. The method of claim 19, wherein said anti-VEGF-C neutralizing antibody material is monoclonal.

21. The method of claim 1, wherein said VEGFR-3 inhibitor down-regulates VEGF-C expression.

22. The method of claim 21, wherein said VEGFR-3 inhibitor is a sequence-specific ribonuclease.

23. The method of claim 22, wherein said sequence-specific ribonuclease is a ribozyme.

24. The method of claim 21, wherein said VEGFR-3 inhibitor is a VEGF-C antisense nucleic acid molecule.

25. The method of claim 1, comprising administering a pharmaceutical composition comprising a cell that secretes said VEGFR-3 inhibitor.

26. The method of claim 1, further comprising administering to said patient an anti-angiogenic agent.

27. The method of claim 1 or claim 26, further comprising administering to said patient an immunosuppressive agent.

28. The method of claim 1, wherein said pharmaceutical composition is administered prior to corneal transplantation.

29. The method of claim 1, wherein said pharmaceutical composition is administered subsequent to corneal transplantation.

30. The method of claim 1, comprising administering to said patient an effective amount of a pharmaceutical composition comprising a VEGFR-3 inhibitor two or more times.

31. The method of claim 30, comprising repeated administration over a period of at least one month.

32. The method of claim 30, comprising repeated administration over a period of at least six months.

33. The method of claim 30, comprising:
(a) administering to said patient prior to corneal transplantation a pharmaceutical composition comprising a VEGFR-3 inhibitor; and
(b) administering to said patient subsequent to corneal transplantation a pharmaceutical composition comprising a VEGFR-3 inhibitor,
whereby lymphangiogenesis is suppressed in the cornea of said patient.

34. The method of claim 1, comprising systemic administration of said pharmaceutical composition.

35. The method of claim 1, comprising local administration of said pharmaceutical composition.

36. The method of claim 35, comprising topical administration of said pharmaceutical composition.

37. The method of claim 35, comprising local injection of said pharmaceutical composition.

38. The method of claim 35, said pharmaceutical composition released from an intraocular or periocular implant.
